(19) United States
(12) Patent Application Publication
(10) Pub. No.: US 2026/0263270 A1
(43) Pub. Date: Sep. 10, 2026

(54) A FLUID DISPENSER SYSTEM AND METHODOLOGY FOR DISTRIBUTING MEDICANTS FOR TREATMENT OF EYE CONDITIONS

(71) Applicant: I IDEAS LLC, Greenport, NY (US)

(72) Inventors: Vincent Basilice, Greenport, NY (US); Dahna Basilice, Greenport, NY (US); Matthew James Ibarra, Cerritos, CA (US)

(21) Appl. No.: 19/560,256

(22) Filed: Mar. 9, 2026

Related U.S. Application Data
(60) Provisional application No. 63/769,108, filed on Mar. 9, 2025.

(51) Int. Cl. A61F 9/00

(52) U.S. Cl. CPC A61F 9/0026

(57) ABSTRACT

A fluid dispenser system comprises a nozzle assembly configured for coupling to a container configured to hold medicants for treatment of one or more conditions of an eye of a subject. The nozzle assembly includes a container coupler segment configured to engage with the container, a medicant chamber segment defining an internal volume configured to receive medicants from the container, a nozzle depending from the medicant chamber segment where the nozzle defines a nozzle passage in fluid communication with the medicant chamber segment and having a nozzle port for distribution of the medicants from the nozzle passage in response to pressurization of the internal volume of the medicant chamber segment, a positioning arm including an engagement segment displaced from the medicant chamber segment where the engagement segment configured to engage at least one of an upper or lower eye lid surrounding the eye to stabilize the one eye lid to facilitate distribution of the medicants into the eye and a medicant seal adjacent the medicant chamber segment and configured to permit passage of the medicants from the container into the internal volume of the medicant chamber segment.

### CROSS-REFERENCE TO RELATED APPLICATION(S)

The present application claims priority to and the benefit of U.S. provisional patent Application Ser. No. 63/769,108, filed Mar. 9, 2025, the entire contents of which disclosure are incorporated herein.

### BACKGROUND

### 1. Technical Field

The present invention is directed to eye dispensers for dispensing medicants in the eye of a subject, and, more particularly, relates to a universal dispenser system and methodology for coupling with a conventional container or bottle to facilitate sterile dispensing of medicants over multiple uses. The present invention is further directed to a universal dispensing system including one or more features adapted to maintain the eye in an open position during disposition of the medicants. The present invention is further directed to a medicant dispensing system including a container apparatus with medicants and a dispenser apparatus couplable to the container apparatus for the sterile delivering of the medicants over one or one or more uses.

### 2. Description of Related Art

Eye droppers are commonly used for dispensing eye solutions or medicants into the eye. There are several types of eye medicants including, without limitation, antibiotic, lubricants, antivirals, anti-inflammatories, antihistamines, glaucoma medications, mydriatics and cycloplegics, immunosuppressants and anesthetics, each intended to treat different conditions affecting the eye. Ensuring accuracy and maintaining the sterility of the medicants in association with applying eye medicants is crucial for their effectiveness over one or more uses.

There are various methods used to assist in maintaining the eye in an open condition and improve the precision of dispensing eye drops. One methodology includes tilting the head of the subject back and maintaining the eye in an open condition with one or more fingers engaging the lower and/or upper eye lids. Other methodologies include auto-drop dispensers such as conical dispensing guide devices which minimize the potential of blinking of the eye during dispensing.

Maintaining the sterility of an eye drop solution over multiple uses is essential to prevent contamination and infections. Traditional approaches include avoiding contact with the tip of the dropper, maintaining the bottle cap tightly closed, washing hands before use and avoid sharing of drops.

Current known and available dispensers have shortcomings which detract from their overall usefulness in dispensing sterile eye medicants, particularly, over multiple uses. The head tilt approach makes self-application difficult and may prevent the user from achieving proper eye treatment using eye drops. Subjects with limited dexterity such as seniors face additional obstacles in eye medicant dispensing. Moreover, current prior art dispensers may also contribute to the spreading of eye disease or infections or interfere with eye treatment.

In addition, conventional systems are subject to contamination when used, either through entry of contaminants within the dispenser during use and/or contact of the dispenser with the eye or any other body portion, which results in undesired contamination of the stored medicants. Moreover, conventional eye distribution systems may be subject to contamination through direct contact of the dropper with the eye and/or surrounding areas, backflow or suction drawing the medicant back into the nozzle when the bottle is released, airborne contaminants, handling contamination via fingers or other surfaces, etc.

### SUMMARY

Accordingly, the present invention relates a fluid or medicant distribution system which addresses the issues associated with conventional systems such as the associated difficulties in distributing drops in the eyes and contamination concerns.

In accordance with one illustrative embodiment, a fluid dispenser system comprises a nozzle assembly configured for coupling to a container configured to hold medicants for treatment of one or more conditions of an eye of a subject. The nozzle assembly includes a container coupler segment configured to engage with the container, a medicant chamber segment defining an internal volume configured to receive medicants from the container, a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment, a positioning arm including an engagement segment displaced from the medicant chamber segment, the engagement segment configured to engage at least one of an upper or lower eye lid surrounding the eye to stabilize the one eye lid to facilitate distribution of the medicants into the eye and a reciprocating nozzle valve at least partially disposed in the nozzle passage of the nozzle, the reciprocating nozzle valve configured to selectively move in the nozzle port to selectively permit and prevent flow of medicants through the nozzle passage and out the nozzle port.

In embodiments, the reciprocating nozzle valve is configured to move in a first direction toward the nozzle port in response to pressurization of the internal volume of the medicant chamber segment to control flow of the medicants through the nozzle port, and in a second direction away from the nozzle port to close the nozzle passage in response to release of pressurization of the internal volume of the medicant chamber segment.

In some embodiments, a cannulated nozzle component is disposed in the nozzle passage of the nozzle for receiving the reciprocating nozzle valve, and permits movement of the reciprocating nozzle valve.

In other embodiments, the reciprocating nozzle valve and the cannulated nozzle component define a passageway therebetween during movement of the reciprocating nozzle valve in the first direction to permit passage of the medicants out the nozzle port.

In embodiments, the reciprocating nozzle valve and the cannulated nozzle component include cooperating structure to close the passageway during movement of the reciprocating nozzle valve in the second direction.

In some embodiments, the cooperating structure include respective tapered portions of the reciprocating nozzle valve and the cannulated nozzle component.

In other embodiments, a spring is disposed in the nozzle passage for biasing the reciprocating nozzle valve in the second direction to close the nozzle passage.

In embodiments, the reciprocating nozzle valve includes outer grooves, where the outer grooves permit passage of the medicants through the nozzle port when the reciprocating nozzle valve is moved in the first direction.

In some embodiments, an air inlet port in fluid communication with the medicant chamber segment to enable ingress of air into the medicant chamber segment.

In other embodiments, a filter in communication with the air inlet port for filtering air.

In embodiments, a reciprocating pressurization valve is at least partially disposed in an air inlet passage of the air inlet port, the reciprocating pressurization valve configured to selectively move in the air inlet port to selectively permit and prevent flow of air into the medicant chamber segment and relative to the container.

In some embodiments, a cannulated pressurization component is disposed in the air inlet port for receiving the reciprocating pressurization valve, and permits movement of the reciprocating pressurization valve.

In other embodiments, the reciprocating pressurization valve and the cannulated pressurization component define a passageway therebetween during movement of the reciprocating nozzle valve in a first direction to permit entry and passage of air though the air inlet port.

In embodiments, the reciprocating pressurization valve and the cannulated pressurization component include cooperating structure to close the passageway during movement of the reciprocating pressurization valve in a second direction.

In some embodiments, the cooperating structure include respective tapered portions of the reciprocating pressurization valve and the cannulated pressurization component.

In other embodiments, a medicant seal is disposed between the internal volume of the medicant chamber segment and the container.

In embodiments, the medicant seal includes one or more seal passages to permit passage of fluids therethrough.

In some embodiments, a first seal passage of the medicant seal is configured to permit passage of the medicants into the internal volume of the medicant chamber segment and a second seal passage of the medicant seal is configured to permit passage of air into the container to facilitate equalization of pressure inside the container.

In other embodiments, the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

In embodiments, the medicant chamber segment, the nozzle and the container coupler segment are a monolithic unit.

In some embodiments, the nozzle and the container coupler segment are a monolithic unit, and is coupled to the medicant chamber segment.

In other embodiments, the positioning arm is configured to depend from one of the medicant chamber segment, the nozzle or the container coupler segment, the positioning arm being selectively movable relative to the medicant chamber segment to deform the medicant chamber segment to pressurize the internal volume to facilitate distribution of the medicants through the nozzle passage and out the nozzle port.

In accordance with another illustrative embodiment of the present invention, a fluid dispenser system comprises a nozzle assembly configured for coupling to a container configured to hold medicants for treatment of one or more conditions of an eye of a subject, The nozzle assembly includes a container coupler segment configured to engage with the container, a medicant chamber segment defining an internal volume configured to receive medicants from the container, a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment, a positioning arm including an engagement segment displaced from the medicant chamber segment whereby the engagement segment is configured to engage at least one of an upper or lower eye lid surrounding the eye to stabilize the one eye lid to facilitate distribution of the medicants into the eye, a medicant seal adjacent the medicant chamber segment, the medicant seal permitting passage of the medicants from the container into the internal volume of the medicant chamber segment and an air inlet port configured to enable entry of air into the medicant chamber segment.

In embodiments, a filter is in communication with the air inlet port for filtering air entering into the container.

In some embodiments, the positioning arm is configured to depend from one of the medicant chamber segment, the nozzle or the container coupler segment, the positioning arm being selectively movable relative to the medicant chamber segment to deform the medicant chamber segment to impart pressure into the internal volume to facilitate distribution of the medicants through the nozzle passage and out the nozzle port.

In other embodiments, the medicant seal includes one or more seal passages to permit passage of fluids therethrough.

In embodiments, a first seal passage of the medicant seal is configured to permit passage of the medicants into the internal volume of the medicant chamber segment and a second seal passage of the medicant seal is configured to permit passage of air into the container to facilitate equalization of pressure inside the container.

In some embodiments, the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

In other embodiments, a reciprocating nozzle valve at least partially disposed in the nozzle passage of the nozzle, the reciprocating nozzle valve configured to selectively move in the nozzle port between an open condition to permit passage of the medicants out the nozzle port and a closed condition to selectively permit flow of medicants through the nozzle port.

### BRIEF DESCRIPTION OF THE DRAWINGS

FIGS. 1 and 2 are perspective and partial perspective views respectively of a fluid dispenser system for distributing medicants for treatment of eye conditions in accordance with one or more illustrative embodiments of the present disclosure;

FIG. 3 is a partial perspective view in cross-section of the fluid dispenser system of FIGS. 1 and 2 in accordance with one or more illustrative embodiments of the present disclosure;

FIG. 4 is a flow chart illustrating an exemplative methodology of use of the fluid dispenser system of FIGS. 1 and 2 in accordance with one or more illustrative embodiments of the present disclosure;

FIG. 5 is a perspective view of another fluid dispenser system in accordance with one or more illustrative embodiments of the present disclosure;

FIG. 6 is a perspective view in cross-section of the fluid dispenser system of FIG. 5 in accordance with one or more illustrative embodiments of the present disclosure;

FIGS. 7 and 8 are perspective and cross-sectional views of a medicant seal of the fluid dispenser system of FIGS. 5 and 6 in accordance with one or more illustrative embodiments of the present disclosure;

FIGS. 9 and 10 are perspective views of another fluid dispenser system in accordance with one or more illustrative embodiments of the present disclosure;

FIGS. 11 and 12 are partial perspective cross-sectional and side cross-sectional views respectively of the fluid dispenser system of FIGS. 9 and 10 in accordance with one or more illustrative embodiments of the present disclosure;

FIGS. 13 and 14 are perspective views of a medicant seal of the fluid dispenser system of FIGS. 9-12 in accordance with one or more illustrative embodiments of the present disclosure;

FIGS. 15 and 16 are perspective and cross-sectional views of respectively of another fluid dispenser system in accordance with one or more illustrative embodiments of the present disclosure;

FIG. 17 is a side cross-sectional view of the fluid dispenser system of FIGS. 15 and 16 in accordance with one or more illustrative embodiments of the present disclosure;

FIG. 17A is a side cross-sectional view of the nozzle of the fluid dispenser system of FIGS. 15-17 in accordance with one or more illustrative embodiments of the present disclosure;

FIG. 18 is a perspective view of another fluid dispenser system in accordance with one or more illustrative embodiments of the present disclosure;

FIG. 19 is a perspective view in cross-section of the fluid dispenser system of FIG. 18 in accordance with one or more illustrative embodiments of the present disclosure;

FIG. 20 is an enlarged perspective view in cross-section illustrating details of the lever valve and the air inlet port of the fluid dispenser system of FIGS. 18 and 19 in accordance with one or more illustrative embodiments of the present disclosure;

FIG. 21 is a perspective view of another fluid dispenser system in accordance with one or more illustrative embodiments of the present disclosure;

FIG. 22 is a perspective view in cross-section of the fluid dispenser system of FIG. 21 in accordance with one or more illustrative embodiments of the present disclosure;

FIG. 23 is a perspective view of the reciprocating nozzle valve of the fluid dispenser system of FIGS. 21 and 22 in accordance with one or more illustrative embodiments of the present disclosure;

FIG. 24 is a perspective view of another fluid dispenser system in accordance with one or more illustrative embodiments of the present disclosure;

FIG. 25 is a perspective view in cross-section of the fluid dispenser system of FIG. 24 in accordance with one or more illustrative embodiments of the present disclosure;

FIG. 26 is a perspective view of another fluid dispenser system in accordance with one or more illustrative embodiments of the present disclosure;

FIG. 27 is a perspective view in cross-section the fluid dispenser system of FIG. 26 in accordance with one or more illustrative embodiments of the present disclosure;

FIG. 28 is a perspective view of another fluid dispenser system in accordance with one or more illustrative embodiments of the present disclosure;

FIG. 29 is a perspective view in cross-section the fluid dispenser system of FIG. 28 in accordance with one or more illustrative embodiments of the present disclosure;

FIG. 30 is a partial perspective view illustrating the interior of the fluid dispenser system of FIGS. 28 and 29 in accordance with one or more illustrative embodiments of the present disclosure.

FIG. 31 is a cross-sectional view illustrating the interior of the fluid dispenser system of FIGS. 28-30 in accordance with one or more illustrative embodiments of the present disclosure.

FIG. 32 is a perspective view of another fluid dispenser system in accordance with one or more illustrative embodiments of the present disclosure;

FIG. 33 is a perspective view with portions in phantom of the fluid dispenser system of FIG. 32 in accordance with one or more illustrative embodiments of the present disclosure;

FIG. 34 is a perspective view in cross-section of the fluid dispenser system of FIGS. 32 and 33 in accordance with one or more illustrative embodiments of the present disclosure;

FIG. 35 is an enlarged perspective view of the of the fluid dispenser system of FIGS. 32-34 in accordance with one or more illustrative embodiments of the present disclosure; and

FIG. 36 is an enlarged perspective view of the of the fluid dispenser system of FIGS. 32-35 in accordance with one or more illustrative embodiments of the present disclosure; and

FIG. 37 is a flow chart illustrating an exemplative methodology of use of the fluid dispenser system of FIGS. 32-36 in accordance with one or more illustrative embodiments of the present disclosure;

### DETAILED DESCRIPTION

The present invention is directed to a fluid dispenser system which may be used with one or more container types comprising medicants for treating the eye. The fluid dispenser system includes various features which enable precise deposition of medicants into the eye by stabilizing the eye lid, in embodiments, the lower eye lid during deposition, and selectively controlling the volume of medicants deposited in the eye. The fluid dispenser system includes an arm which depends outwardly and defines an outer surface dimensioned to engage either the upper or lower eye lid to maintain the eye in an open condition. A nozzle depends at an angle relative to the arm. The nozzle includes a nozzle passage for transferring the medicants into the eye while the arm is engaged with the eye lid. A medicant chamber of the fluid dispenser system is in fluid communication with the nozzle. In embodiments, the nozzle includes a reciprocating valve disposed in a nozzle passage of the nozzle. The reciprocating valve moves to selectively open and close the nozzle passage to permit passage of medicants through the nozzle. The medicant chamber may include a medicant seal arranged to control flow of medicants through/from the container and, in embodiments, passage of air into the container to maintain pressure equilibrium within the container. The medicant seal minimizes the potential of medicants leaking back into the container subsequent to use thereby maintaining sterility of the medicants. In embodiments, the arm may move or pivot relative to the medicant chamber to create pressure or pressurize the internal volume of the medicant chamber to direct the medicants out the nozzle. The fluid dispenser system improves dosing of medicants, enhances patient comfort and maintains sterility of the medicants during multiple uses.

In embodiments, at least the reciprocating nozzle and/or the medicant seal and optionally other components of the fluid dispenser system operate in a manner to maintain the sterility of the medicants within the fluid container. In some embodiments, these features enable the provided medicant to be free of any preservatives which are conventionally used in eye drop formulations.

Various embodiments of the adaptable precision fluid medication dispenser system are described in detail below.

In embodiments, the fluid dispenser system includes the fluid dispenser and the container which is coupled to the container. The fluid dispenser and the container may be separate components which are assembled at the operative site. In other embodiments, the fluid dispenser and the container may be coupled and provided to the operative site as an integral unit or apparatus.

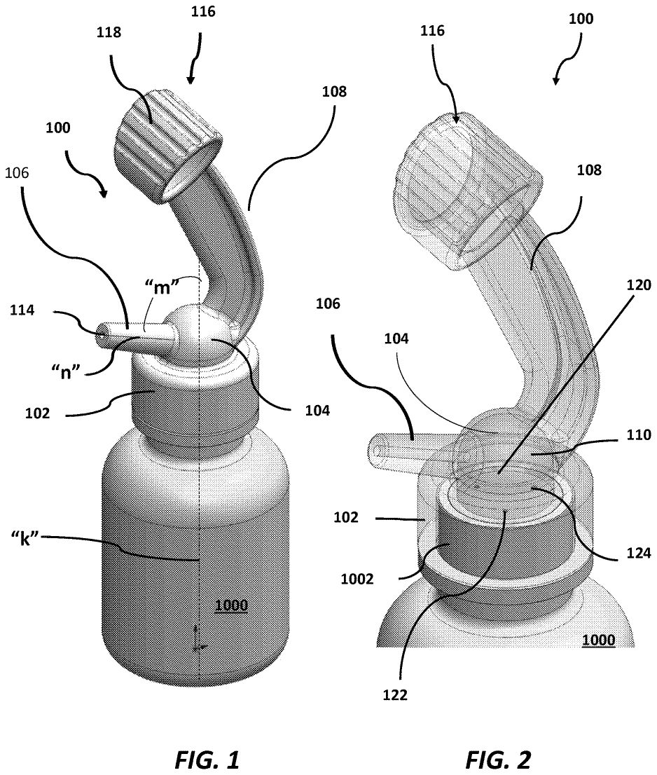

Referring now to FIGS. 1-3, one illustrative embodiment of the fluid dispenser system in accordance with principles of the present invention is disclosed. The fluid dispenser system 100 is adapted to be coupled to a container 1000 of the type containing or holding one or more medicants for treatment of a condition of the eye. The container 1000 may be any conventional bottle, plastic container, vial or the like having structure to enable coupling to the fluid dispenser system 100. The container 1000 may be provided as a component of the fluid dispenser system 100 or may be a separate component secured to the fluid dispenser system 100 at the operative site. The container 1000 defines a longitudinal axis “k”. The container 1000 may be solid or may be flexible in whole or in part. The fluid dispenser system 100 includes a container coupler segment 102, a medicant chamber segment 104, a nozzle 106 depending outwardly from the medicant chamber segment 104 and a positioning arm 108. The container coupler segment 102 includes structure to couple with the container 1000. The structure may include threading or threaded segments, a bayonet coupling, a cam lock or a tolerance surface or the like. In embodiments, the coupler segment 102 defines an internal cylinder which is positioned over a correspondingly dimensioned outlet 1002 of the container 1000 in, for example, frictional engagement therewith to establish a tolerance fit or the like. Cements or adhesives may be used to secure the coupler segment 102 to the container outlet 1002. In other embodiments, the coupler segment 102 may be coupled to the container outlet 1002 via a snap-fit connection or a threaded coupling.

The medicant chamber segment 104 defines an internal volume 110 which receives the medicants from the container 1000. In embodiments, the medicant chamber segment 104 is dome-shaped. Other configurations are also envisioned. In some embodiments, the medicant chamber segment 104 is at least partially deformable to dispense the medicants through the nozzle 106.

The nozzle 106 defines a nozzle axis “n” which extends at an oblique angle relative to the longitudinal axis “k” of the container 1000 to which the fluid dispenser system 100 is coupled. In embodiments, the angle “m” may range between about 15° to about 85° or between about 45° to about 75° or about 60°. The nozzle 106 defines an internal nozzle passage 112 terminating in a nozzle port 114. The positioning arm 108 extends from the medicant chamber segment 104 also at an angle relative to the longitudinal axis “k.” In embodiments, the positioning arm 108 may be arcuate in configuration. The positioning arm 108 is arranged to assist with dispensing the volume of fluid in an eye of a user. The positioning arm 108 includes an engagement segment 116 configured to engage the lower and/or upper eye lid surrounding the eye of the subject. In embodiments, the engagement segment 116 is arcuate or cylindrical in configuration. The engagement segment 116 includes engagement structure 118 which may, in embodiments, include grooves, knurling, surface irregularities, protrusions or the like, which may extend around the entire surface or along a partial surface of the engagement segment 116 are atraumatic to the user. In embodiments, the engagement structure 118 may be capable of gripping the lower eye lid even in the presence of fluids and/or medicants on the eye lid. In other embodiments, the engagement segment 116 defines a smooth surface, may include a coating, etc. to facilitate engagement with the eye lid. In embodiments, the arcuate arrangement of the positioning arm 108 positions the engagement segment 116 in general alignment with the axis “k” of the container 1000. This alignment may facilitate contact and engagement of the engagement segment 116 relative to the eye lid. For example, and without limitation, the container 1000 and the engagement segment 116 may share a common visual axis to enable the user to guide both components simultaneously using the same hand orientation and/or improving fine motor control due to the lack of offset between the components. In some embodiments, the user may perceive the engagement segment 116 and the container 1000 as a single reference structure, i.e., the general coaxial arrangement enhances spatial predictability and is more intuitive.

The fluid dispenser system 100 also includes a medicant seal 120 disposed adjacent to or at the interface of the medicant chamber segment 104 and the container 1000. The medicant seal 120 includes one or more seal passages 122, 124 extending through the membrane of the medicant seal 120. Three seal passages 122, 124 are shown. In one embodiment, the first seal passage(s) 122 is configured to enable passage of the medicants from the container 1000 to the internal volume 110 of the medicant chamber segment 104. In some embodiments, the first seal passage(s) 122 is a one-way valve permitting passage of medicants to the internal volume 110 of the medicant chamber segment 104 while preventing retrograde movement of the medicants from the internal volume 110 back into the container 1000. The second seal passage 124 permits passage of air back into the container 1000 to equalize pressurization within the container 1000 subsequent, e.g., to deposition of the medicants. In embodiments, two seal passages 122 are configured to enable flow of medicants between the container 1000 and the medicant chamber segment 104. In other embodiments, two seal passages 124 are configured to transmit air between the container 1000 and the medicant chamber segment 104. In other embodiments, the three (3) seal passages are configured to minimize or reduce pressure loss in the internal volume 110 of the medicant chamber segment 104 when the positioning arm 108 is deflected. The first and second seal passages 122, 124 operate in conjunction to ensure equilibrium is established within the container subsequent to dispensing of one or more drops. This prevents retrograde movement of the medicant back into the container thereby preserving the contents of the medicant.

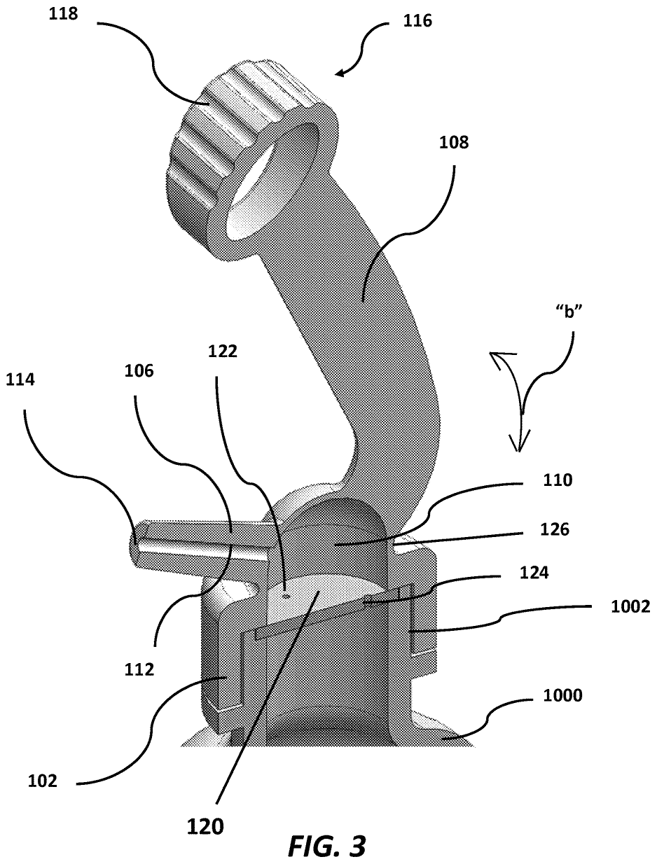

With reference to FIG. 3, the positioning arm 108 may be relatively stable to maintain its orientation relative to the container 1000. In other embodiments, the positioning arm 108 may be configured to move or be movable relative to the medicant chamber segment 104 to deform the medicant chamber segment 104 and impart an internal pressure within the internal volume 110. In some embodiments, the positioning arm 108 may reciprocally pivot along the interface 126 of the positioning arm 108 and the medicant chamber segment 104 in the direction “b” to deform the medicant chamber segment 104. For example, the positioning arm 108 may pivot toward the container 1000 to pressurize the internal volume 110 and return to the position depicted in FIG. 3 upon release of the positioning arm 108. During return movement of the positioning arm 108, air may pass from the internal volume 110 of the medicant chamber segment 104 through the second seal passage 124 to equalize pressurization within the container 1000.

In embodiments, the positioning arm 108 may include a deformable segment to deform and compress the medicant chamber segment 104. In other embodiments, the medicant chamber segment 104 may be directly engaged by the user without deforming or moving the positioning arm 108 to compress the medicant chamber segment 104 to pressurize the internal volume 110. In other embodiments, the container 1000 may be moved toward the user and/or laterally relative to the eye such the positioning arm 108 deforms the medicant chamber segment 104 with or without movement pivoting movement of the positioning arm 108.

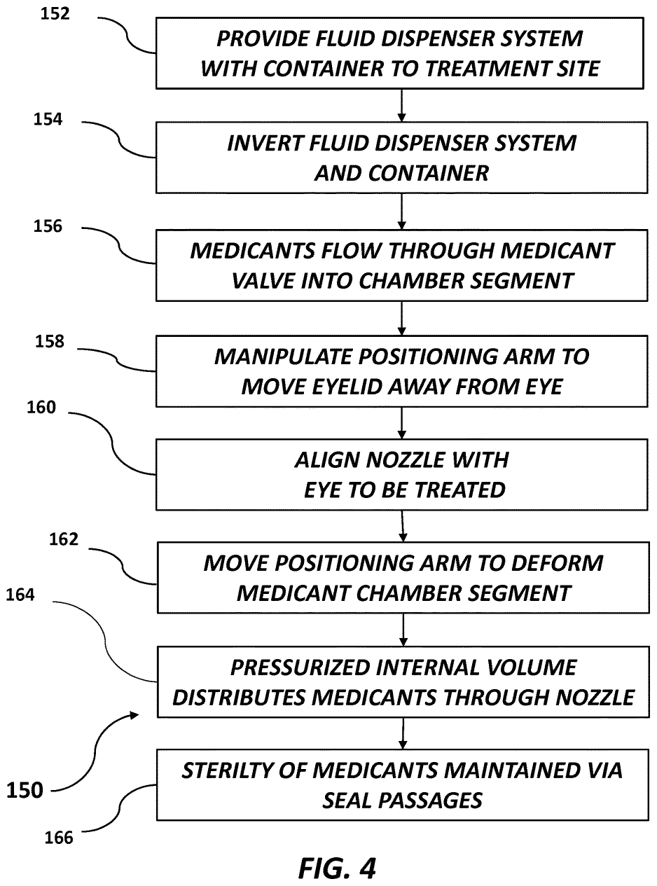

FIG. 4 illustrates a flow chart 150 depicting an illustrative methodology of use of the fluid dispenser system 100 and the container 1000. In use, the fluid dispenser system 100 is coupled to the container 1000, and provided at a treatment site. (STEP 152). In embodiments, the fluid dispenser system 100 and the container 1000 are pre-coupled and delivered to a site in the coupled relation. In other embodiments, the fluid dispenser system 100 is separate from the container 1000 and coupled prior to use. The fluid dispenser system 100 and container 1000 are inverted. (STEP 154). This action causes medicants to flow from the container 1000 through the first seal passage 122 of the medicant seal 120 and into the internal volume 110 of the medicant chamber segment 104. (STEP 156). The positioning arm 108 is gently applied to the eye lid, e.g., the lower eye lid, such that the engagement segment 116 of the positioning arm 108 engages the eyelid. The container 1000 is manipulated to cause the positioning arm 108 to move the lower eye lid downwardly away from the eye to create a distribution area under the eye. (STEP 158). In embodiments, the distribution area may be a small space or pocket between the lower lid and the eye surface, e.g., the conjunctival sac. During movement, the engagement structure 118 of the engagement segment 116 maintains its grip on the eyelid. In some embodiments, the positioning arm 108 may pivot to move the lower eye lid. In some embodiments, the positioning arm 108 may pivot toward the nozzle 106. In other embodiments, the positioning arm 108 may be stable relative to the container 1000 to move the lower eye lid without pivoting movement of the positioning arm 108. Due to the angular arrangement of the nozzle 106 and the positioning arm 108, the nozzle may be positioned in alignment with the lower eye area created by the displaced eye lid. The nozzle 106 is positioned in alignment with the small space or pocket of the eye to be treated. (STEP 160). The positioning arm 108 is moved or pivoted by manipulation of the container 1000 to cause the positioning arm 108 to deform the medicant chamber segment 104 while still maintaining engagement of the engagement structure 118 with the eye lid. (STEP 162). Deformation of the medicant chamber segment 104 pressurizes the internal volume 110 to cause the medicants to exit the nozzle passage 112 and the nozzle port 114 for entry into the eye of the subject. (STEP 164). In some embodiments, the positioning arm 108 may pivot slightly to align the nozzle with the pocket and further pivoting movement of the positioning arm 108 causes deformation of the medicant chamber segment 104 to distribute the medicants. More specifically, the pivoting arm 108 may pivot through a first predetermined angle of deformation without causing deformation of the medicant chamber segment 104 to create the pocket. Further pivoting movement of the positioning arm 108 through a second predetermined angle of deformation beyond the first predetermined angle of deformation will cause deformation of the medicant chamber segment 104 to cause distribution of the medicants through the nozzle port 114. In other embodiments, pivoting movement of the positioning arm 108 will cause deformation of the medicant chamber segment 104 and distribution of the medicants from the nozzle port 114. In other embodiments, the positioning arm 108 may be stable relative to the container 1000 and moved relative to the container 1000 to create the pocket. Thereafter, the container 1000 may be manipulated by pressing, pulling, moving the container laterally etc. to cause the positioning arm 108 to cause deformation of the medicant chamber segment 104 and distribution of the medicants through the nozzle 106. During use, the sterility of the medicants is maintained at least in part to the equalizes functionalities of the seal passages 122, 124, the arrangement of the nozzle 106 and the positioning arm 108. (STEP 166) For example, and without limitation, the seal passages 122, 124 operate in concert to prevent or minimize draw-back of medicants into the container 1000. In embodiments, the nozzle passage 112 and the nozzle port 114 are configured to dispense one or more drops of medicants approximately 5 microliters (μl) to approximately 60 μl. or about 30 μl per drop.

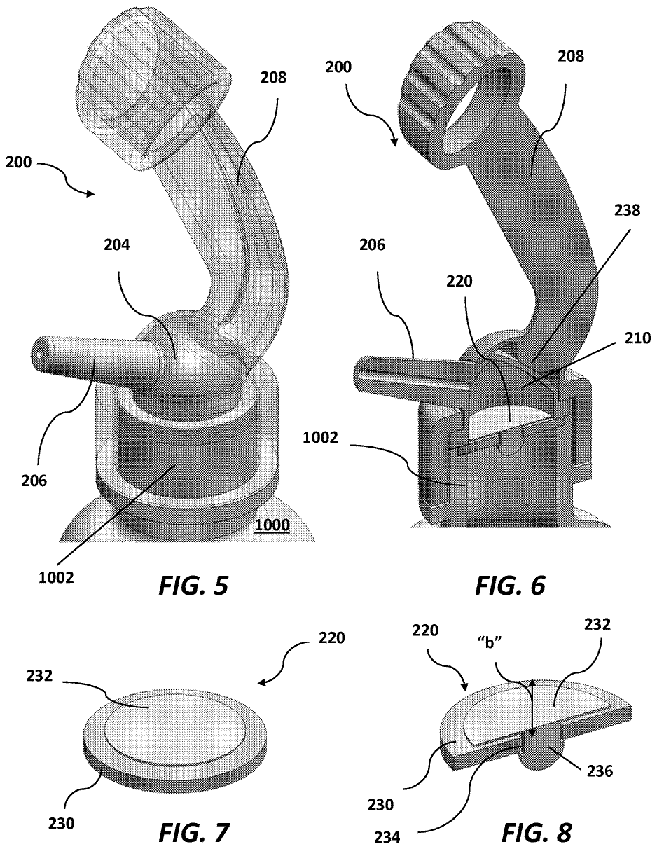

Referring now to FIGS. 5-8, another illustrative embodiment of the fluid dispenser system in accordance with the principles of the present invention is disclosed. The fluid dispenser system 200 is similar in some respects to the fluid dispenser system 100. In this embodiments, the container coupler segment 202 and the positioning arm 208 are monolithically formed as a first unit and the medicant chamber segment 204 and the nozzle 206 are monolithically formed as a second unit. The first unit is mounted about or over the second unit, and coupled by conventional means. The fluid dispenser system 200 further includes a medicant seal 220 which is different in some respects to the medicant seal 120 of FIGS. 1-4. In particular, the medicant seal 220 includes a first membrane 230 and a second membrane 232 disposed on the first membrane 230 in superposed relation. The first membrane 230 defines a central aperture 234. The second membrane 232 includes a closure flap 236 which is at least partially received in the central aperture 234 of the first membrane 230 in a normal condition of the medicant seal 220. The first membrane 230 may reciprocally move in the directions “b” during use of the fluid dispenser system 200. For example, upon inversion of the container 1000 and the fluid dispenser system 200, the second membrane 232 is displaced relative to the first membrane 230 whereby the closure flap 236 opens the central aperture 234 of the first membrane 230 to enable passage of medicants into the medicant chamber segment 204. When the positioning arm 208 is moved, deflected or pivoted, a chamber compressing segment 238 of the positioning arm 208 engages an outer surface of the medicant chamber segment 204 to deform the medicant chamber segment 204 and pressurize the internal volume 210 of the medicant chamber segment 204. The pressurized internal volume drives the second membrane 232 against the first membrane 230 to cause the closure flap 236 to be at least partially or fully received in the central aperture 234 to close the central aperture 234. In this position, the potential of ingress of medicants from the internal volume 210 of the medicant chamber segment 204 back into the container 1000 is minimized or eliminated thereby preserving the sterility of the medicants.

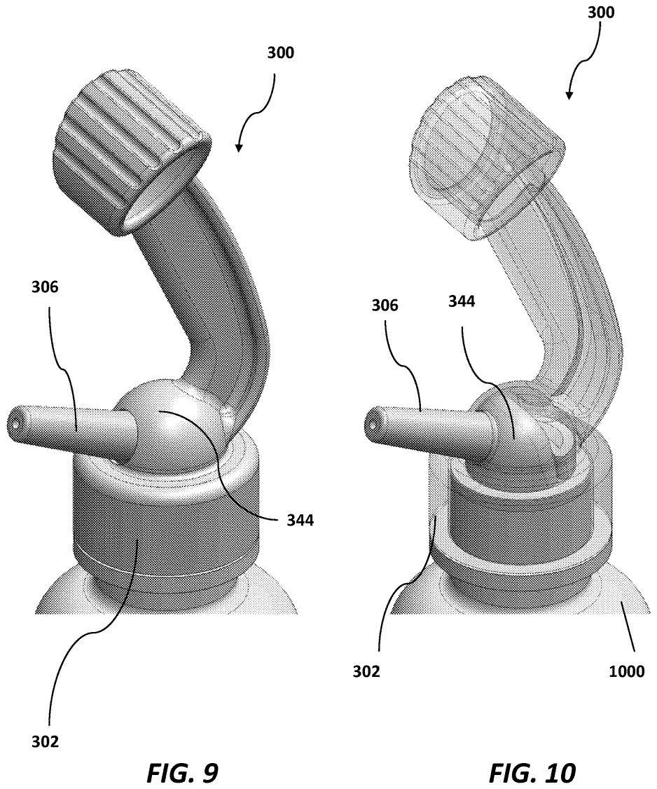

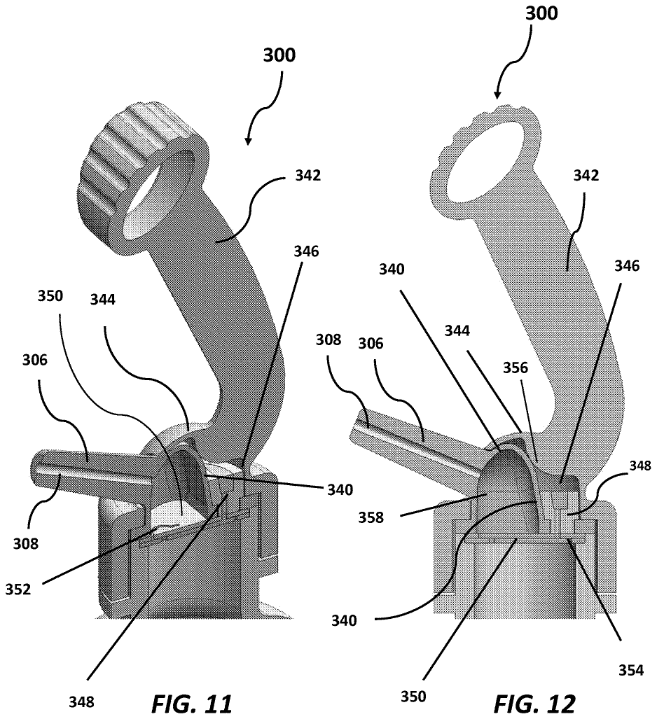

Referring now to FIGS. 9-12, another illustrative embodiment of the fluid dispenser system in accordance with the principles of the present invention is disclosed. The fluid dispenser system 300 includes a container coupler segment 302 and a positioning arm 342 depending from the container coupler segment 302. The container coupler segment 302 and a positioning arm 342 are a single, e.g., monolithically formed unit. The fluid dispenser system 300 further includes a nozzle 306 and a medicant chamber segment 340 which may be formed as a single monolithic unit. The monolithic units are coupled to each other via conventional means. The fluid dispenser system 300 further includes a medicant chamber segment 340 which is integrally formed with the nozzle 306. (FIGS. 7 and 8). A positioning arm 342 extends from the coupler segment 302. The positioning arm 342 includes an arcuate segment 344 at least partially disposed around the medicant chamber segment 340 to define an internal air chamber 346. In some embodiments, the internal air chamber 346 is not air tight. For example, air may pass through the nozzle passage 308 of the nozzle 306. An air filter 348 is disposed in the internal air chamber 346. The fluid dispenser system 300 further includes a medicant seal 350 at the juncture of the coupler segment 302 and the container outlet 1002 of the container 1000. The medicant chamber segment 340 and the internal air chamber 346 are in fluid communication with the container 1000 through the medicant seal 350.

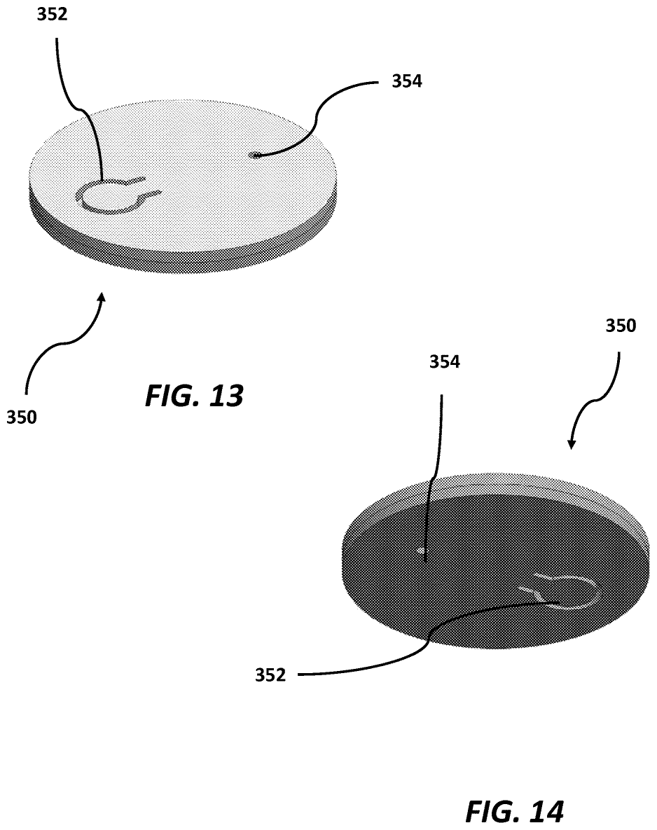

With reference to FIGS. 13 and 14, in conjunction with FIGS. 9-12, the medicant seal 350 includes a first seal passage 352 in fluid communication with the medicant chamber segment 340 and a second seal passage 354 in fluid communication with the internal air chamber 346. The first seal passage 352 may include an arcuate slit or passage and the second seal passage 354 may be an aperture. Other configuration are also envisioned. The first seal passage 352 permits flow of medicants from the container 1000 into the medicant chamber segment 340 upon conversion of the container 1000 and restricts or eliminates retrograde flow of the medicants back into the container 1000. The second seal passage 354 permits air to flow from the internal air chamber 346 through the air filter 348 and back into the container 1000 to equalize pressure within the container 1000. The air filter 348 filters any contaminants which may be present to maintain the sterility of the medicant within the container 1000.

In use, the fluid dispenser system 300 and the container 1000 are inverted to permit passage of the medicants from the container 1000 through the first seal passage 352 and into the medicant chamber segment 340. The positioning arm 342 is arranged relative to the eye with the engagement structure 318 engaging the lower eye lid. The positioning arm 342 is pivoted or moved whereby internal actuating portion 356 of the positioning arm 342 compresses or deforms the medicant chamber segment 340 to pressurize the internal volume 358 and dispense a dose of medicants through the nozzle passage 312 and out the nozzle port 314. Upon release of the positioning arm 342, air is conveyed from the internal air chamber 346 through the air filter 348 and the second seal passage 354, and into the container 1000 to equalize pressurization within the container 1000.

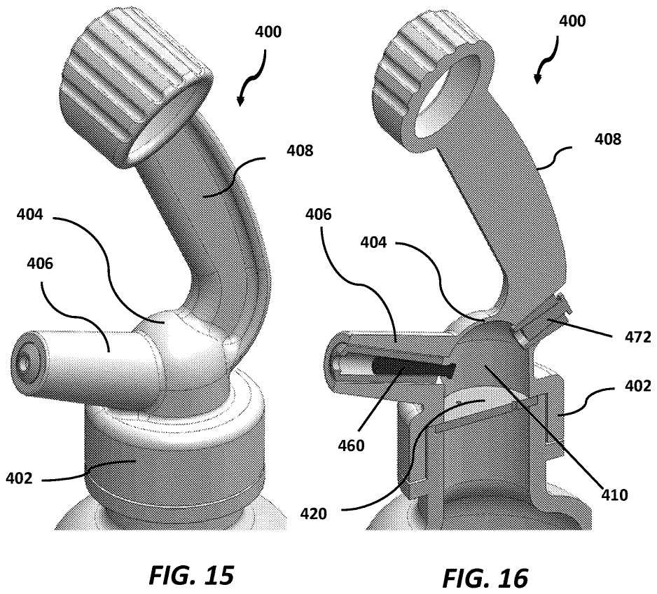

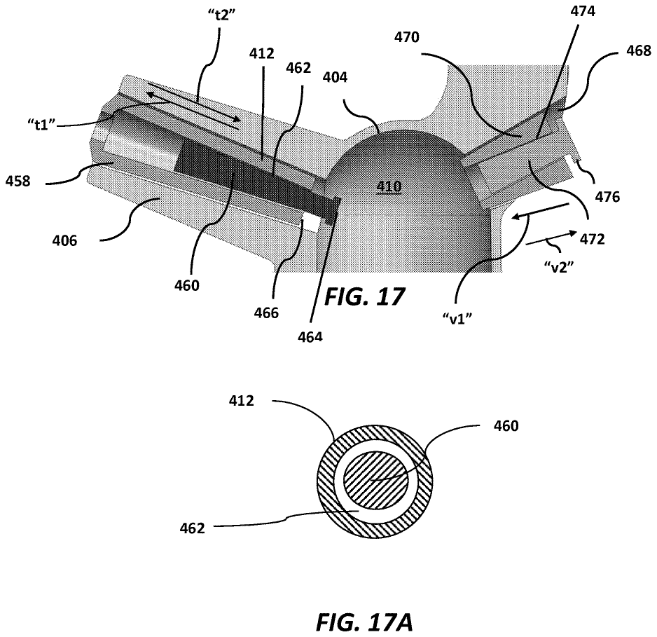

Returning now to FIGS. 15-17 and 17A, one or more illustrative embodiments of the present invention is depicted. The fluid dispenser system 400 includes a coupler segment 402, a medicant chamber segment 404, a nozzle 406 and a medicant seal 420. The nozzle 406 defines a nozzle passage 412 which increases in diameter to the nozzle port 114. A cannulated nozzle component 458 is disposed within the nozzle 406. Disposed within the nozzle component 458 is a reciprocating nozzle valve 460. The reciprocating nozzle valve 460 is configured to selectively reciprocally move in the nozzle component 458 to selectively permit flow of medicants from the medicant chamber segment 404 and through the nozzle 406. In embodiments, the reciprocating nozzle valve 460 is configured to move in a first direction “t1” toward the nozzle port 414 in response to pressurization of the internal volume 410 of the medicant chamber segment 404 to control flow of the medicants through the nozzle port 414. In some embodiments, the reciprocating nozzle valve 460 and the cannulated nozzle component 458 define a passageway 462 therebetween (around the outer surface of the reciprocating nozzle valve 460) to enable passage of the medicants when advanced in the first direction “t1” as depicted in FIG. 17A. In some embodiments, the cannulated nozzle component 458 defines an internal taper and the reciprocating nozzle valve 460 defines a corresponding outer taper. During movement of the reciprocating nozzle valve 460 toward the nozzle port 414 in response to deformation of the medicant chamber segment 404, medicants flow around the exterior of the reciprocating nozzle valve 460 (through the passage 462 defined between the reciprocating nozzle valve 460 and the internal surface of the cannulated nozzle component 458) and out the nozzle port 414 until such time the back stop 464 of the reciprocating nozzle valve 460 engages the internal surface 466 of the cannulated nozzle component 458. Upon release of the positioning arm 408, the medicant chamber segment 404 returns to its original configuration (responsive to the medicant chamber segment 404 being equalized with air, e.g., under vacuum) which causes the reciprocating nozzle valve 460 to move away from the nozzle port 414 in the second direction “t2”. In embodiments, during return movement of the reciprocating nozzle valve 460, an enlarged outer tapered portion of the reciprocating nozzle valve 460 engages a relatively smaller inner tapered portion of the nozzle component 458 to close the cannulated nozzle component 458 thereby preventing release of medicants through the nozzle 406.

The fluid dispenser system 400 further includes an air inlet port 468 in fluid communication with the medicant chamber segment 404 and a cannulated pressurization component 470 disposed in the air inlet port 468. A reciprocating pressurization valve 472 is at least partially disposed in the cannulated pressurization component 470, and reciprocally moves in the directions “v1”, “v2”. The reciprocating pressurization valve 472 is configured to selectively move in the air inlet port 468 to selectively permit and, optionally, prevent flow of air into the medicant chamber segment 404 and relative to the container 1000. In embodiments, the cannulated pressurization component 470 defines an internal dimension or diameter which gradually increases toward the medicant chamber segment 404. Similarly, the reciprocating pressurization valve 472 defines an outer diameter dimension which gradually increases toward the medicant chamber segment 404. An air passageway 474 is defined between the inner surface of the cannulated pressurization component 470 and the outer surface of the reciprocating pressurization valve 472 to permit air to flow into the medicant chamber segment 404—similar to the action of the reciprocating nozzle valve 460 and the cannulated nozzle component 458 depicted in FIG. 17A. In use, the positioning arm 108 is manipulated to deform the medicant chamber segment 404 to dispense the medicants through the nozzle 106. Upon return of the positioning arm 408 to its normal position, a vacuum is formed in the internal volume 410 of the medicant chamber segment 404 which draws air from the outside into the air inlet port 468 (e.g., through the passageway 474) and the medicant chamber segment 104. Concurrently therewith, the reciprocating pressurization valve 472 moves toward the medicant chamber segment 404 in the direction “v1” until the back stop 476 engages the outer surface of the cannulated pressure component 470. In this position, the air inlet port 468 is closed preventing further air to enter therein. In addition, or in the alternative, an enlarged outer tapered portion of the reciprocating pressurization valve 472 engages a relatively smaller inner tapered portion of the cannulated pressurization component 470 to seal or close the cannulated nozzle component 458 thereby preventing entry/release of air relative to the medicant chamber segment 404. The air is communicated through one of the seal passages 424 of the medicant seal 420 to equalize pressure in the container 1000. When equilibrium is established the reciprocating pressurization valve 472 moves in the direction “v2” which causes the outer tapered surface of the reciprocating valve 472 to engage the internal surface of the cannulated pressurization component 470 to close the cannulated pressure component 470 preventing ingress of air into the medicant chamber segment 404.

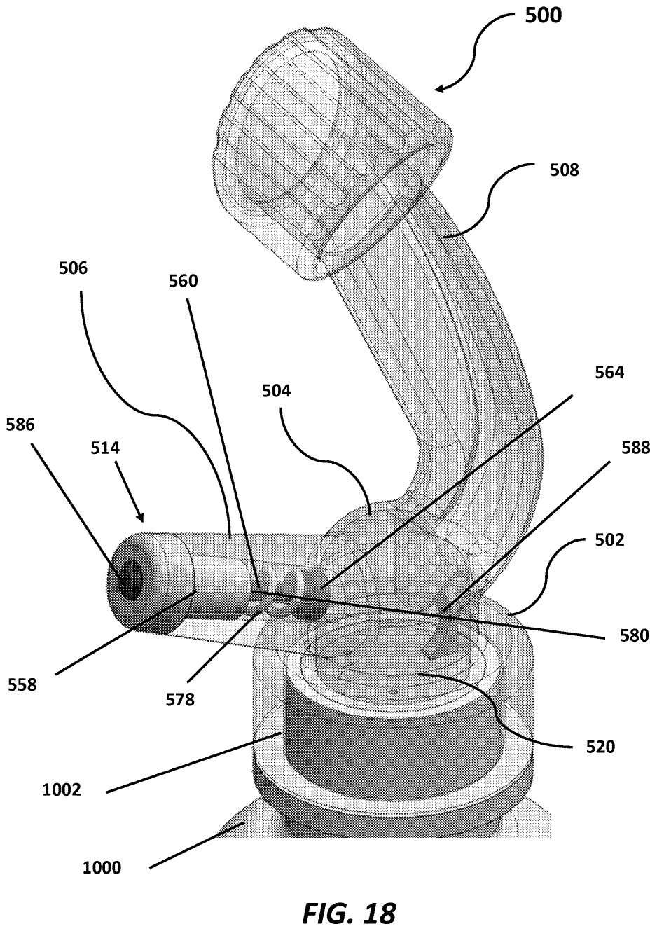

Referring now to FIGS. 18-20, one or more illustrative embodiments of the present invention is depicted. The fluid dispenser system 500 includes a coupler segment 502, a medicant chamber segment 504, a nozzle 506 and a medicant seal 520. The nozzle 506 defines a nozzle passage 512 which increases in diameter to the nozzle port 514. A cannulated nozzle component 558 is disposed within the nozzle 506. Disposed within the nozzle component 558 is a reciprocating nozzle valve 560. The reciprocating nozzle valve 560 is configured to selectively move in the nozzle component 558 to selectively permit flow of medicants from the medicant chamber segment 504 and through the nozzle 506. In embodiments, the reciprocating nozzle valve 560 is configured to move in a first direction toward the nozzle port 514 in response to pressurization of the internal volume 510 of the medicant chamber segment 504 to control flow of the medicants through the nozzle port 514. In some embodiments, the reciprocating nozzle valve 560 and the cannulated nozzle component 558 is capable of defining a passageway therebetween (around the outer surface of the reciprocating nozzle valve 560) to enable passage of the medicants when the reciprocating nozzle valve 560 is advanced toward the nozzle port 514. In some embodiments, the cannulated nozzle component 558 defines an internal taper portion and the reciprocating nozzle valve 560 defines a corresponding outer taper portion to create the passageway.

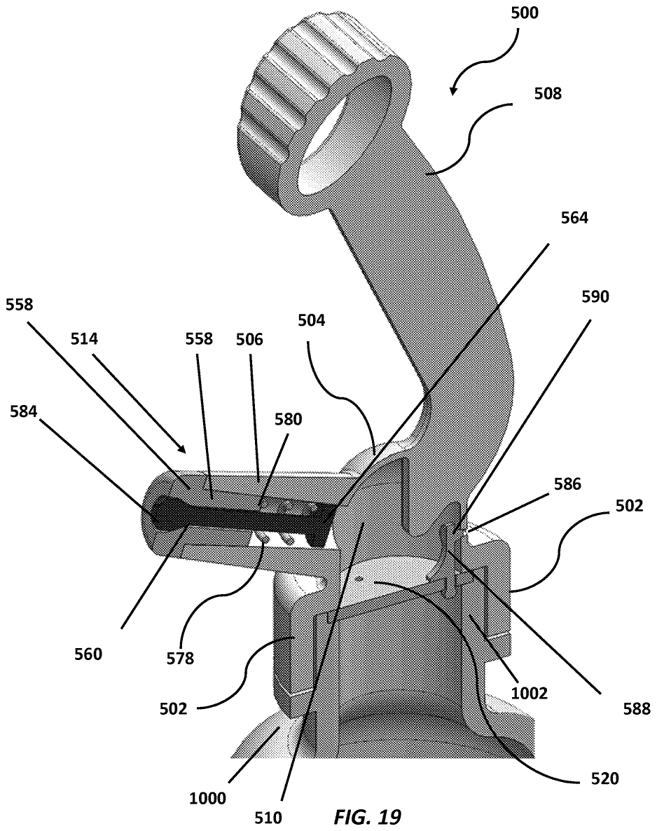

The fluid dispenser system 500 further includes a coil spring 578 coaxially arranged along a portion of the reciprocating nozzle valve 560. The coil spring 578 normally biases the reciprocating nozzle valve 560 toward the medicant chamber segment 504 corresponding to a closed condition of the nozzle 506. The coil spring 578 engages a proximal surface 580 of the cannulated nozzle component 558 and a back stop 564 of the reciprocating nozzle valve 560. The back stop 564 closes the nozzle passage 512 of the nozzle 506 when in a normal at rest position of the condition of the reciprocating nozzle valve 560 depicted in FIG. 19. Alternatively, or in addition, the reciprocating nozzle valve 560 may include a distal closure element 584 dimensioned to also close the cannulated nozzle component 558 when in an at rest position of the reciprocating nozzle valve 560.

In some embodiments, the cannulated nozzle component 558 defines an internal taper and the reciprocating nozzle valve 560 defines a corresponding outer taper. During movement of the reciprocating nozzle valve 560 toward the nozzle port 514 in response to deformation of the medicant chamber segment 504 and pressurization of the internal volume 510, medicants flow around the exterior of the reciprocating nozzle valve 560 (through a passage defined between the reciprocating nozzle valve 560 and the internal surface of the cannulated nozzle component 558) and out the nozzle port 514 until. In some embodiments, the back stop 564 of the reciprocating nozzle valve 560 may engage the internal surface 580 of the cannulated nozzle component 558. Upon release of the positioning arm 508, the medicant chamber segment 504 returns to its original configuration under the bias of coil spring 578 which causes the reciprocating nozzle valve 560 to move away from the nozzle port 514 to close the nozzle 506. In embodiments, during return movement of the reciprocating nozzle valve 560, an enlarged outer tapered portion of the reciprocating nozzle valve 560 engages a relatively smaller inner tapered portion of the nozzle component 558 to close the cannulated nozzle component 458 thereby preventing release of medicants through the nozzle 506. In addition, or in the alternative, the distal closure element 584 of the reciprocating nozzle valve 560 will close the nozzle 506.

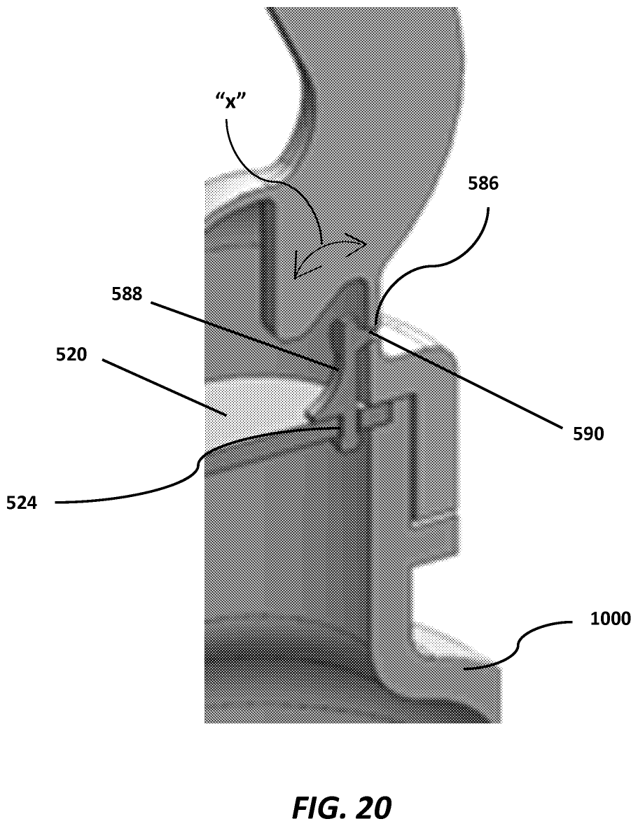

As best depicted in the enlarged view of FIG. 20, in conjunction with FIGS. 18 and 19, the fluid dispenser system 500 further includes an air inlet port 586 to permit passage of the air into the medicant chamber segment 504. A lever valve 588 is mounted to one of the seal passages 524 and includes a lever valve segment 590 which is received within the air inlet port 586 in the at rest position of the lever valve 588 shown in FIG. 20. The lever valve 588 is adapted to reciprocally pivot (shown by directional arow “x”) between the closed position with the lever valve segment 590 received within the air inlet port 586 (FIG. 20) and an open condition with the lever valve segment 590 displaced from the air inlet port 586 to permit entry of air into the internal volume 510. In embodiments, the lever valve 588 pivots about a fulcrum defined at the juncture of the lever valve segment 590 and the medicant seal 520.

In embodiments, the positioning arm 508 is manipulated to deform the medicant chamber segment 504 to dispense the medicants through the nozzle 506. Upon return of the positioning arm 508 to its normal position, a vacuum is formed in the medicant chamber segment 504 which draws air from the outside into the air inlet port 586 to cause the lever valve 588 to pivot to displace the lever valve segment 590 from the air inlet port 586 to enable passage of air into the internal volume 510 of the medicant chamber segment 504 to equalize pressure within the medicant chamber segment 504. The air also will pass through one of the seal passages 524 of the medicant seal 220 into the container 1000 to equalize pressure within the container 1000. Once pressure equilibrium is established within the medicant chamber segment 504, the lever valve 588 returns to the original position depicted in FIGS. 18-20 to close the air inlet port 586.

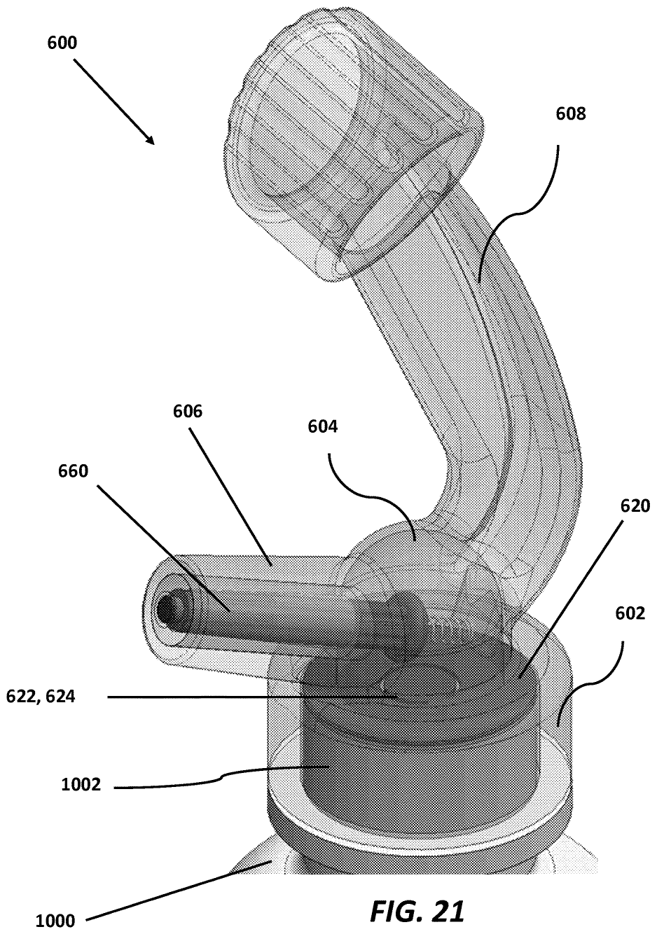

FIGS. 21-23 illustrate another embodiment of the fluid dispenser system of the present invention. The fluid dispenser system 600 includes a coupler segment 602, a medicant chamber segment 604, a nozzle 606 and a medicant seal 620. A cannulated nozzle component 658 is disposed within the nozzle 506. Disposed within the nozzle component 658 is a reciprocating nozzle valve 660. The reciprocating nozzle valve 660 is configured to selectively reciprocally move in the nozzle component 658 to selectively permit flow of medicants from the medicant chamber segment 604 and through the nozzle 606. In embodiments, the reciprocating nozzle valve 660 is configured to move in a first direction away from the nozzle port 614 in response to pressurization of the internal volume 610 of the medicant chamber segment 604 to control flow of the medicants through the nozzle port 614. In some embodiments, the reciprocating nozzle valve 660 and the cannulated nozzle component 658 are capable of defining a passageway therebetween (around the outer surface of the reciprocating nozzle valve 660) to enable passage of the medicants. In some embodiments, the cannulated nozzle component 658 defines an internal taper portion and the reciprocating nozzle valve 660 defines a corresponding outer taper portion.

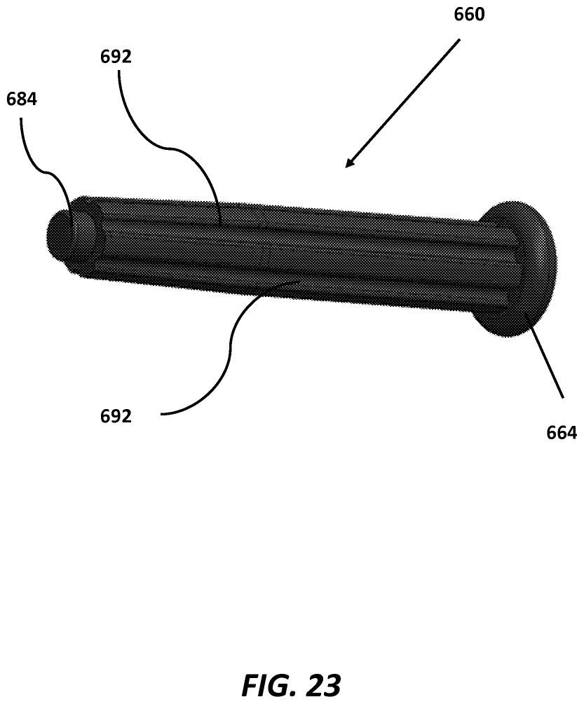

As best depicted in FIG. 23, the reciprocating nozzle valve 660 defines a plurality of longitudinal grooves 692 extending along a major portion or the entire portion of the length of the reciprocating nozzle valve 660. The longitudinal grooves 692 provide conduits to enable the medicants to flow out the nozzle 606. The reciprocating nozzle valve 660 further includes a distal closure element 684 which is received within the cannulated nozzle component 658 in the normal condition of the reciprocating nozzle valve 660 to close the nozzle passage of the cannulated nozzle component 658.

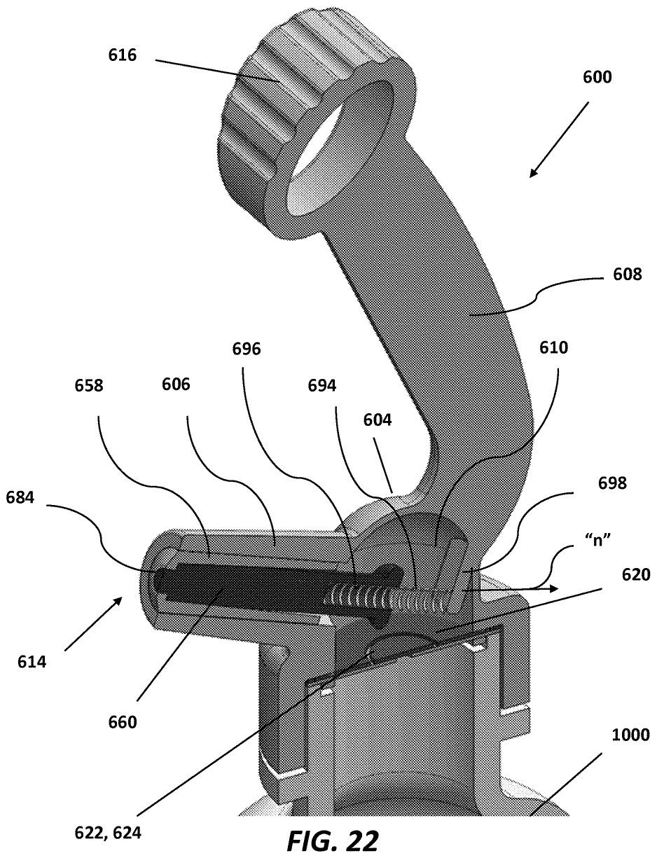

The fluid dispenser system 600 further includes a coil spring 694 at least partially disposed within a recess 696 of the reciprocating nozzle valve 660. The coil spring 694 engages an internal spring stop 698 associated with the medicant chamber segment 604 or the positioning arm 608. The coil spring 694 biases the reciprocating nozzle valve 660 toward the nozzle port 614 corresponding to a closed condition of the cannulated nozzle component 658 depicted in FIG. 22. In other embodiments, the coil spring 694 is secured to the reciprocating nozzle valve 660 at the leading end of the coil spring 694 and secured to the internal spring stop 698 at the other trailing end of the coil spring 694 to normally bias the reciprocating the nozzle valve 660 in a proximal direction corresponding to an open condition of the reciprocating nozzle valve 660. In other embodiments, the proximal bias of the coil spring 694 corresponds to the closed condition of the reciprocating nozzle valve 660. The coil spring 694 may be fabricated from metallic or polymeric materials.

The medicant seal 620 defines an annular seal passage 622/624 which may be complete or partial, e.g., a complete circle or a partial circle. The annular seal passage 622/624 enables both passage of medicants from the container 1000 and the longitudinal grooves 692 of the nozzle valve 660 and out the nozzle port 614.

In use, the container 1000 and attached fluid dispensing system 600 is inverted to cause flow of medicants from the container 1000 through the annular seal passage 622/624 into the medicant chamber segment 604. The positioning arm 608 is positioned to engage the engagement segment 616 with the lower eye lid. The container 1000 is manipulated to cause the positioning arm 608 to deform the medicant chamber segment 604. Deformation of the medicant chamber segment 604 increases the internal pressure within the internal volume 610 of the medicant chamber segment 604 to distribute the fluids through the longitudinal grooves 692 of the reciprocating nozzle valve 660 and out the nozzle port 614. In embodiments, the internal pressurization and/or the accumulation of fluids within the internal volume 610 will draw the reciprocating nozzle valve 660 away from the nozzle port 614 against the bias of coil spring 694 to open and/or further open the nozzle port 614 and distribution of fluids from the nozzle port 614. In other embodiments, pivoting movement of the positioning arm 608 moves the internal spring stop 698 in the direction “n” which enables the reciprocating nozzle valve 660 to move away from the nozzle port 614. (via, e.g., altering or reducing the spring constant of the coli spring 694). In other embodiments, the pressurized fluid forces the head 664 of the reciprocating nozzle valve 660 toward the internal spring stop 698 to open the reciprocating nozzle valve 660. Movement of the reciprocating nozzle valve 660 causes displacement of the distal closure element 684 from the nozzle port 614 to open the nozzle passage 612 of the reciprocating nozzle valve 660 and/or permit passage of the medicants at least through the longitudinal grooves 692 of the reciprocating nozzle valve 660. The medicants are dispensed through the nozzle port 614. Upon release of the displacing force applied to the positioning arm 608, the positioning arm 608 and the medicant chamber segment 604 returns to their normal conditions. With the internal pressure removed from the internal volume 610, the reciprocating nozzle valve 660 returns to its normal condition under the influence of the coil spring 694 to close the nozzle port 614. In embodiments, air within the internal volume 610 of the medicant chamber segment 604 passes through the seal passage 622/624 to equalize pressure within the container 1000. In other embodiments, there is no passage of air through the seal passages 622/624.

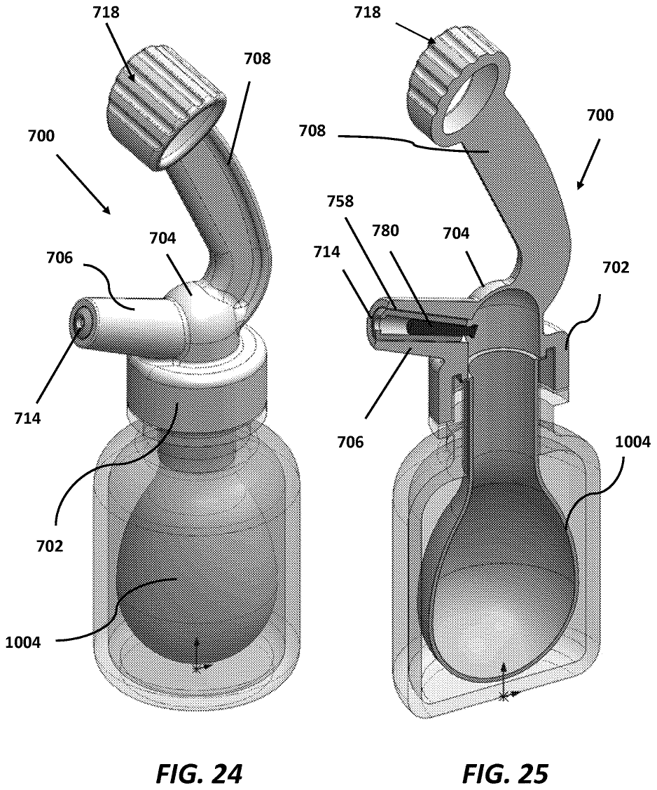

FIGS. 24 and 25 illustrate another embodiment of the fluid dispenser system of the present invention. The fluid dispenser system 700 includes a coupler segment 702, a medicant chamber segment 704, and a nozzle 706. The nozzle includes a cannulated nozzle component 758 with a reciprocating nozzle valve 780 disposed therein. The reciprocating nozzle valve 780 may be similar in design and function to any of the reciprocating nozzle valves described hereinabove. The container 1000 includes a flexible bladder 1004 which contains the medicants. In embodiments, the fluid dispenser system 700 is devoid of a medicant seal separating the container 1000 and the fluid dispenser system 700. In use, the container 1000 and the fluid dispenser system 700 are inverted and the engagement structure 718 of the positioning arm 708 is arranged to engage the lower eye lid. The container 1000 is manipulated to bend or flex the positioning arm 708 to deform the medicant chamber segment 704 to dispense the medicants through the nozzle 706 and the nozzle port 714 by virtue of actuation of the reciprocating nozzle valve 780. After distribution of the medicants, the container 1000 is manipulated to return the positioning arm 708 to its normal state. In embodiments, the flexible bladder 1004 is fully enclosed within the container 1000. The flexible bladder 1004 may collapse during distribution of the fluids. In embodiments, there is no re-entry of air into the flexible bladder 1004 after the positioning arm 708 resets upon release from the eyelid.

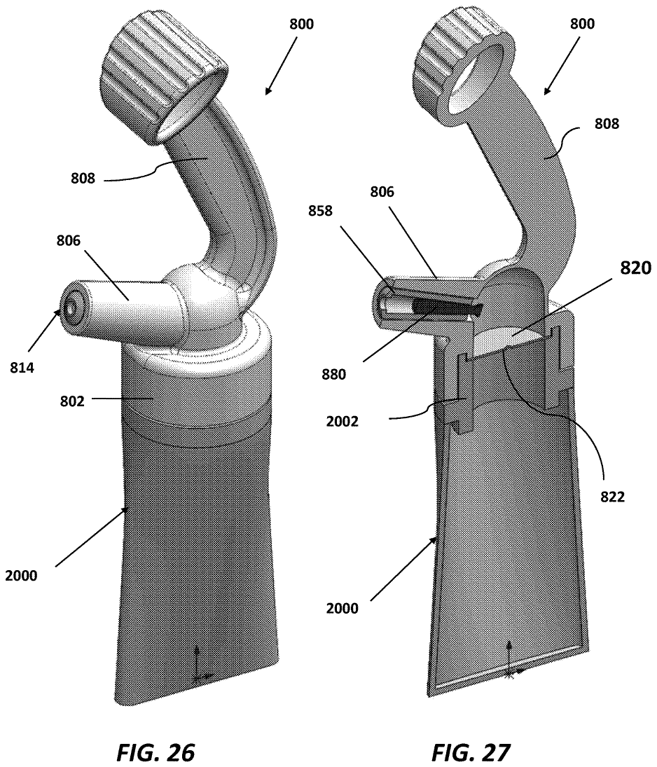

FIGS. 26 and 27 illustrate another embodiment of the fluid dispenser system of the present invention. The fluid dispenser system 800 includes a coupler segment 802, a medicant chamber segment 804, a nozzle 806 and a medicant seal 820. The nozzle 806 includes a cannulated nozzle component 858 with a reciprocating nozzle valve 880 disposed therein. The reciprocating nozzle valve 880 may be similar in design and function to any of the reciprocating nozzle valves described hereinabove. The medicant seal 820 may be similar in design and function to any of the medicant seals described hereinabove. The container 2000 includes a container outlet 2002 for coupling to the container coupler segment 802 of the fluid dispenser system 800. The container 2000 may be a flexible sleeve in whole or in part. The flexible sleeve may, in embodiments, be squeezed or compressed to distribute medicants through the medicant seal 820 and into the medicant chamber segment 804. In other embodiments, the flexible sleeve may be inverted to permit passage of the medicants through the seal passage 822 via gravity. The positioning arm 808 may be pivoted to expel the medicants through deformation of the medicant chamber segment 804 to dispense the medicants through the nozzle 806 and the nozzle port 814 by virtue of actuation of the reciprocating nozzle valve 880. After distribution of the medicants, the container 2000 is manipulated to return the positioning arm 808 to its normal state. In embodiments, there is no re-entry of air into the container 2000. The container 2000 collapses in sequence or progression after, for example, each use.

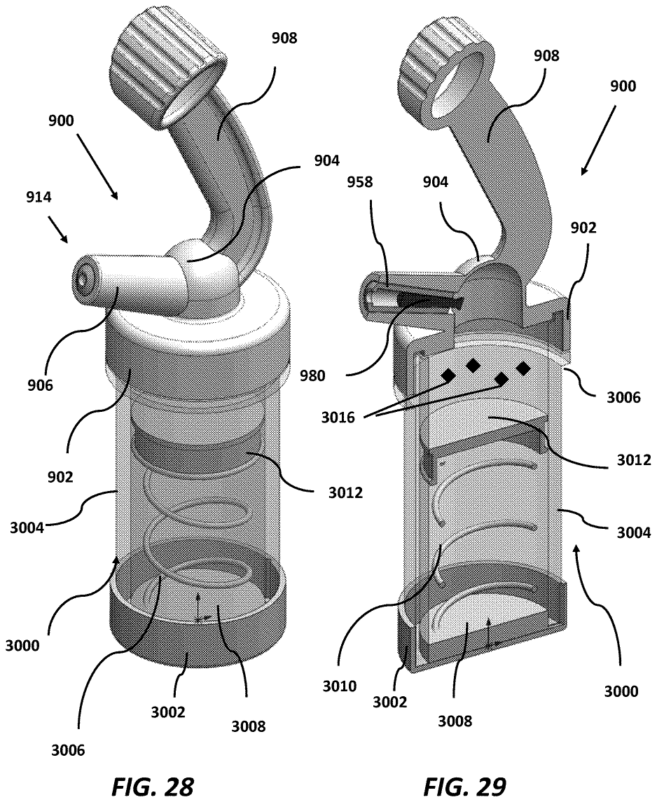

FIGS. 28-31 illustrate another embodiment of the fluid dispenser system of the present invention. The fluid dispenser system 900 includes a coupler segment 902, a medicant chamber segment 904 and a nozzle 906. The nozzle 906 includes a cannulated nozzle component with a reciprocating nozzle valve 980 disposed therein. Each of these components may be similar in design and function to any of the like components described hereinabove. The container 3000 includes a container base 3002, a container body 3004 and a container outlet 3006 for coupling to the container coupler segment 902 of the fluid dispenser system 900. The container base 3002 and the container body 3004 may be separate components coupled to each other or a single monolithic component.

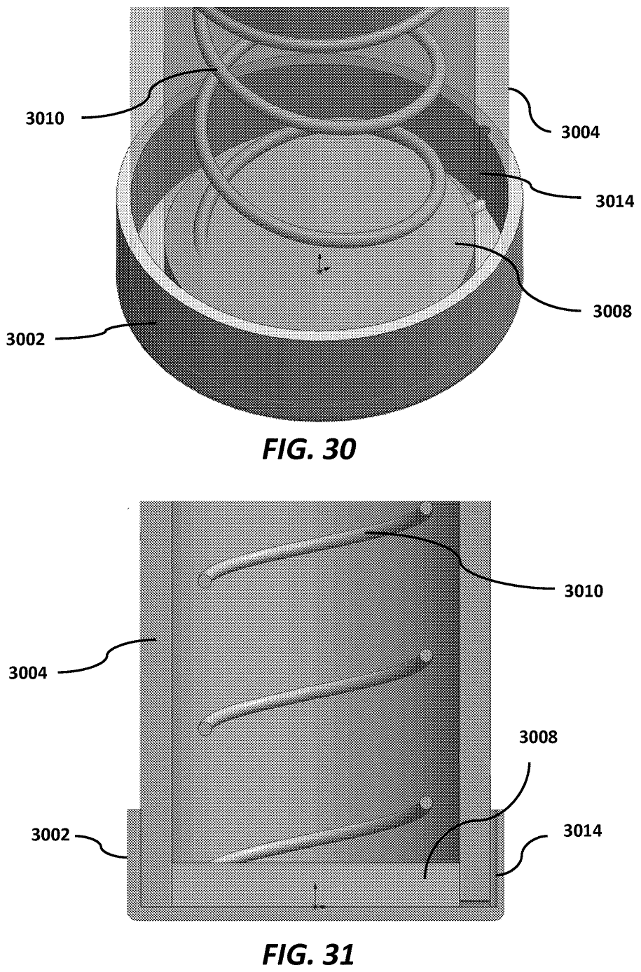

As best depicted in FIGS. 30 and 31, in conjunction with FIGS. 28 and 29, the container 3000 further includes a hydrophobic filter 3008, a coil or compression spring 3010 extending from the hydrophobic filter 3008 and a platform 3012 disposed above and mechanically coupled to the coil spring 3010. The hydrophobic filter 3008 may be disc-shaped, and may be made from materials such as PTFE (polytetrafluoroethylene), polypropylene, or other water-repelling polymers. A channel inlet 3014 is disposed within the container to enable air to enter and pass through the hydrophobic filter 3008. In embodiments, the channel inlet 3014 is defined between the container base 3002 and the container body 3004. In embodiments, the channel inlet 3014 may be an axial bore extending through the container base 3002 or may be a side bore extending through either the container base 3002 or the container body 3004. The channel inlet 3014 is in fluid communication with the hydrophobic filter 3008 to filter air entering the container body 3004 during re-pressurization of the container body 3004.

The platform 3012 may be sealed relative to the interior surface of the container 3000. The platform 3012 is configured to move within the container 3000 toward the medicant chamber segment 904. The platform 3012 is biased in the upward direction toward the medicant chamber segment 904 under the pressure influence exerted by the coil spring 3010 to drive the medicant, represented schematically as reference numeral 3016, into the medicant chamber segment 904. In embodiments, the spring force of the coil spring 3010 may be sufficient to actuate the reciprocating nozzle valve 980 to dispense the medicant into the eye. In other embodiments, or in addition, the medicant is dispensed through inversion of the container 3000 and deformation of the a medicant chamber segment 904 via actuation or pivoting of the positioning arm 908 as described hereinabove. For example, the positioning arm 908 may be pivoted to expel the medicants 3016 through deformation of the medicant chamber segment 904 to dispense the medicants through the nozzle 906 and the nozzle port 914 by virtue of actuation of the reciprocating nozzle valve 980. After use, and/or upon release of the positioning arm 908, air passes through channel inlet 3014 through hydrophobic filter 3008 and into the container body 3004 to pressurize the interior of the container body 3004.

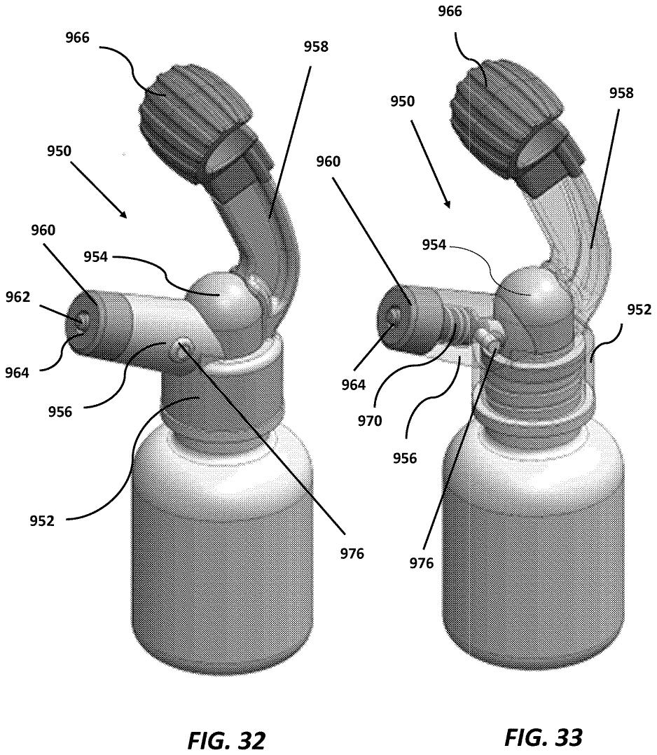

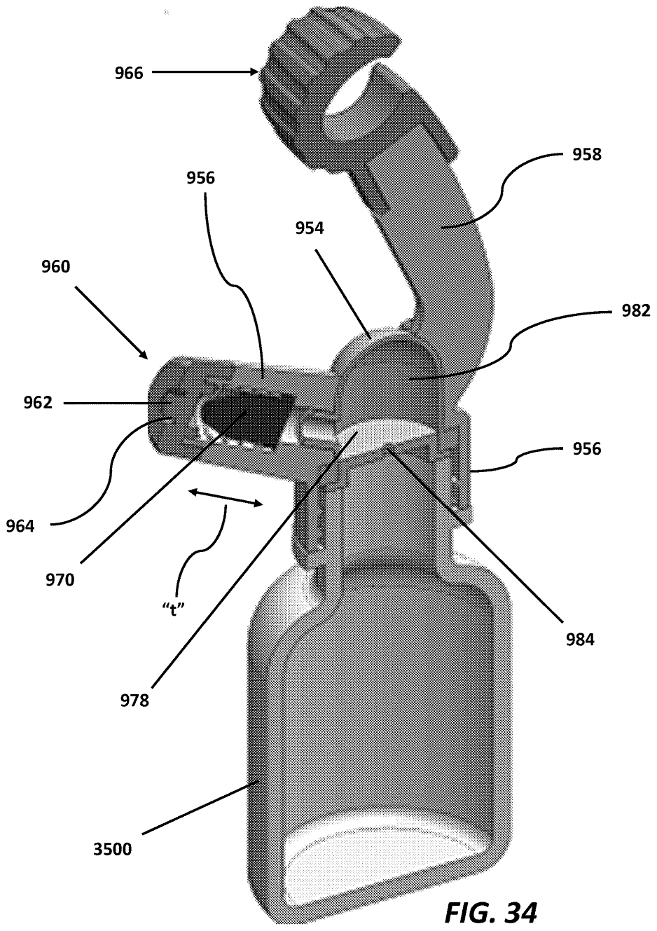

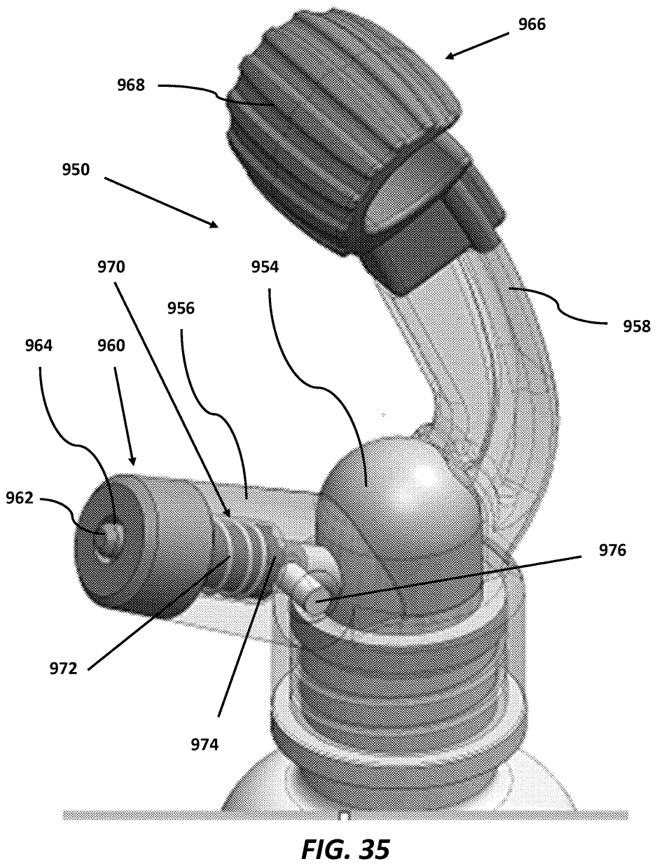

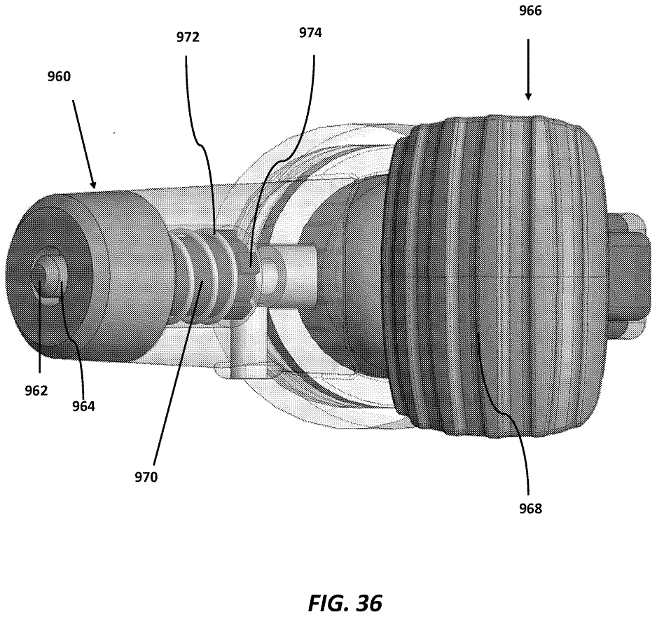

FIGS. 32-36 illustrate another embodiment of the fluid dispenser system of the present invention. The fluid dispenser system 950 includes a coupler segment 952, a medicant chamber segment 954, a nozzle 956 and a positioning arm 958. The nozzle 956 includes a nozzle tip 960 coupled thereto. The nozzle tip 960 includes a central nozzle segment 962 surrounded by an at least partial annular tip passage or port 964. The nozzle tip port 964 permits flow of the fluids from the medicant chamber segment 952 out the nozzle 956. In embodiments, the nozzle tip port 964 is a complete annular channel surrounding the central nozzle segment 962. Other arrangements for the nozzle tip port 964 are envisioned. The nozzle tip 960 may be a separate component from the nozzle 956, and coupled via conventional mechanical and/or adhesive methodologies. In embodiments, the nozzle tip 960 comprises an elastomer which may be soft relative to the nozzle 956.

The positioning arm 958 includes an eyelid contacting segment 966. The arm contacting segment 966 includes multiple ribs, grooves and/or irregularities 968 to facilitate engagement with the eyelid (e.g., the lower eyelid). In embodiments, the eyelid contacting segment 966 is a separate component from the positioning arm 958 and coupled thereto via mechanical and/or adhesive methodologies. The eyelid contacting segment 966 may comprise a soft elastomer or the like.

The fluid dispenser system 950 further includes a reciprocating valve 970 disposed within the nozzle 956. The reciprocating valve 970 may reciprocally move (in the direction of directional arrows “t”) between the closed position depicted in FIGS. 32-34 to an open or advanced position. The reciprocating valve 970 is configured to selectively move in the nozzle 956 to selectively permit flow of medicants from the medicant chamber segment 954 and through the nozzle 956. In embodiments, the reciprocating valve 970 is configured to move in a first direction toward the nozzle tip 960 in response to pressurization of the internal volume 982 of the medicant chamber segment 954 to control flow of the medicants through the nozzle tip port 964. In some embodiments, the reciprocating valve 970 and the nozzle 956 is capable of defining a passageway therebetween (around the outer surface of the reciprocating valve 970) to enable passage of the medicants when the reciprocating valve 970 is advanced toward the nozzle tip 960. A coil spring 972 is coaxially mounted about the reciprocating valve 970 and normally biases the reciprocating valve 970 to the closed position. The distal end of the coil spring 972 may engage the interior of the nozzle 956 (e.g., an internal shelf) of the nozzle 956. The proximal end of the coil spring 972 engages one or outer flanges 974 on the outer surface of the reciprocating valve 970. With this arrangement, the coil spring 972 normally biases the reciprocating valve 970 to the closed position.

The fluid dispenser system 950 further includes a filter 976 extending through the outer surface of the nozzle 956 in communication with the nozzle passage of the nozzle 956. The filter may be an antibacterial filter comprising a microfiber glass or a non-woven fabric (polyethylene, polyester or nylon) and may be infused with antimicrobial elements such as silver. In embodiments, the filter 976 may be a one way filter permitting movement of air in a first direction into the nozzle 956 and restricting flow out the nozzle 956. In some embodiments, the filter 976 is disposed in a channel or passage of the main chamber segment 954. Other locations for the filter 976 are also envisioned including with the container 3500.

In some embodiments, the coupler segment 952, the medicant chamber segment 954, the nozzle 956 and the positioning arm 958 may be separate components coupled to each other via any of the aforementioned methodologies. In some embodiments, at least some of the components are monolithically formed.

The fluid dispenser system 950 also includes a medicant seal 978 disposed adjacent to or at the interface of the medicant chamber segment 954 and the container 3500. The medicant seal 978 includes one or more seal passages 984 extending through the membrane of the medicant seal 978. The seal passage 984 permits flow of the fluids from the container 3500 to the internal volume 982 of the medicant chamber segment 954.

The coupler segment 952 may be coupled to the container 3500 through a threaded coupling including respective cooperating threads within the coupler segment 952 and on the container 3500.

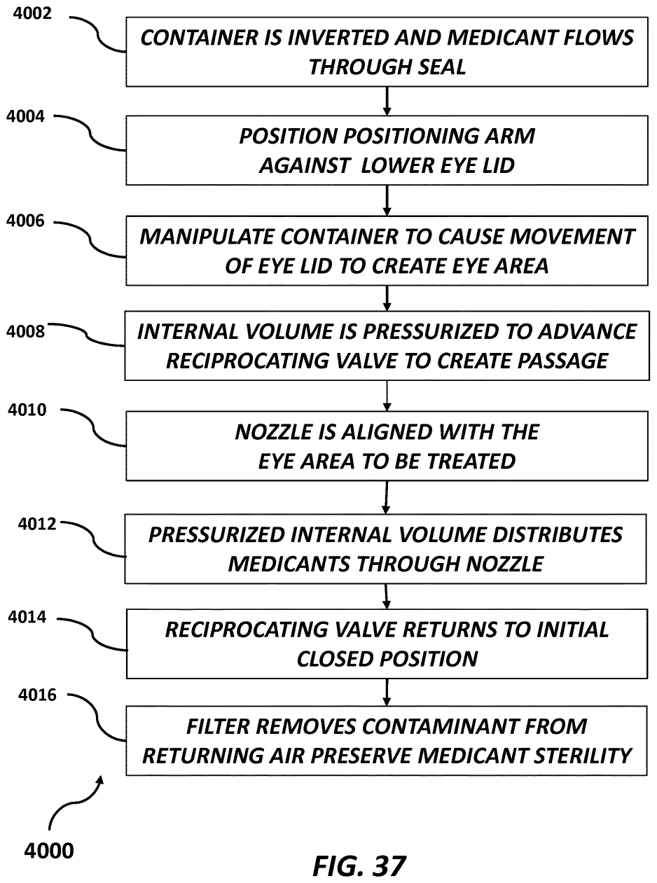

In use, and with reference to the flow chart process 4000 of FIG. 37, the container 3500 is inverted to cause fluids to flow through the seal passage of the medicant seal 978. (STEP 4002) The eyelid contacting segment 966 of the positioning arm 958 is positioned against the lower eyelid (STEP 4004). The container 3500 is manipulated to cause the lower eyelid to move downward away from the eye to open the eye area (STEP 4006). In some embodiments, the positioning arm 958 will move or angulate toward the nozzle 956 to move the lower eye lid. In association therewith, angulation of the positioning arm 958 will cause deformation of the medicant chamber segment 954 and increase pressure within the internal volume to cause the reciprocating valve 970 to advance against the bias of the coil spring 972 toward the nozzle tip port 964. Movement of the reciprocating valve 970 causes unseating of the reciprocating valve 970 relative to the internal shelf of the nozzle 956 and opening of the nozzle passage. (STEP 4008). The nozzle 956 is aligned with the open eye area created by the displaced lower eye lid (STEP 4010). In embodiments, this automatically occurs when the positioning arm 958 is angulated. More specifically, the angular relationship of the positioning arm 958 and the nozzle 956 automatically arranges and/or positions the nozzle in alignment with the open eye area when the container is manipulated 3500. Fluids flow through the nozzle 956 and out the nozzle port 964 of the nozzle tip 960 due to pressurization of the internal volume. (STEP 4012). Upon return of the positioning arm 958 to its normal position, a vacuum is formed in the medicant chamber segment 954 which draws air from the outside through the filter 976 to re-pressurize the medicant chamber segment 954 and cause return of the reciprocating valve to its initial closed condition. (STEP 4014) The filter 976 removes contaminants from the entering air thereby preserving the sterility of the medicants. (STEP 4016).

In other embodiments, the positioning arm is not deformed and the medicants are dispensed through gravity upon inversion of the container 3500.

### I. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A fluid dispenser system, which comprises:
a nozzle assembly configured for coupling to a container configured to hold medicants for treatment of one or more conditions of an eye of a subject, the nozzle assembly including:
a container coupler segment configured to engage with the container;
a medicant chamber segment defining an internal volume configured to receive medicants from the container;
a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment;
a positioning arm including an engagement segment displaced from the medicant chamber segment, the engagement segment configured to engage at least one of an upper or lower eye lid surrounding the eye to stabilize the one eye lid to facilitate distribution of the medicants into the eye; and
a reciprocating nozzle valve at least partially disposed in the nozzle passage of the nozzle, the reciprocating nozzle valve configured to selectively move in the nozzle port to selectively permit and prevent flow of medicants through the nozzle passage and out the nozzle port.

Clause 2. The fluid dispenser system wherein the reciprocating nozzle valve is configured to move in a first direction toward the nozzle port in response to pressurization of the internal volume of the medicant chamber segment to control flow of the medicants through the nozzle port, and in a second direction away from the nozzle port to close the nozzle passage in response to release of pressurization of the internal volume of the medicant chamber segment.

Clause 3. The fluid dispenser system including a cannulated nozzle component disposed in the nozzle passage of the nozzle for receiving the reciprocating nozzle valve, and permitting movement of the reciprocating nozzle valve.

Clause 4. The fluid dispenser system wherein the reciprocating nozzle valve and the cannulated nozzle component define a passageway therebetween during movement of the reciprocating nozzle valve in the first direction to permit passage of the medicants out the nozzle port.

Clause 5. The fluid dispenser system wherein the reciprocating nozzle valve and the cannulated nozzle component include cooperating structure to close the passageway during movement of the reciprocating nozzle valve in the second direction.

Clause 6. The fluid dispenser system wherein the cooperating structure include respective tapered portions of the reciprocating nozzle valve and the cannulated nozzle component.

Clause 7. The fluid dispenser system including a spring disposed in the nozzle passage for biasing the reciprocating nozzle valve in the second direction to close the nozzle passage.

Clause 8. The fluid dispenser system wherein the reciprocating nozzle valve includes outer grooves, the outer grooves permitting passage of the medicants through the nozzle port when the reciprocating nozzle valve is moved in the first direction.

Clause 9. The fluid dispenser system including an air inlet port in fluid communication with the medicant chamber segment to enable ingress of air into the medicant chamber segment.

Clause 10. The fluid dispenser system including a filter in communication with the air inlet port for filtering air.

Clause 11. The fluid dispenser system including a reciprocating pressurization valve at least partially disposed in an air inlet passage of the air inlet port, the reciprocating pressurization valve configured to selectively move in the air inlet port to selectively permit and prevent flow of air into the medicant chamber segment and relative to the container.

Clause 12 The fluid dispenser system including a cannulated pressurization component disposed in the air inlet port for receiving the reciprocating pressurization valve, and permitting movement of the reciprocating pressurization valve.

Clause 13. The fluid dispenser system wherein the reciprocating pressurization valve and the cannulated pressurization component define a passageway therebetween during movement of the reciprocating nozzle valve in a first direction to permit entry and passage of air though the air inlet port.

Clause 14. The fluid dispenser system wherein the reciprocating pressurization valve and the cannulated pressurization component include cooperating structure to close the passageway during movement of the reciprocating pressurization valve in a second direction.

Clause 15. The fluid dispenser system wherein the cooperating structure include respective tapered portions of the reciprocating pressurization valve and the cannulated pressurization component.

Clause 16. The fluid dispenser system including a medicant seal disposed between the internal volume of the medicant chamber segment and the container.

17. The fluid dispenser system wherein the medicant seal includes one or more seal passages to permit passage of fluids therethrough.

Clause 18. The fluid dispenser system wherein a first seal passage of the medicant seal is configured to permit passage of the medicants into the internal volume of the medicant chamber segment and a second seal passage of the medicant seal is configured to permit passage of air into the container to facilitate equalization of pressure inside the container.

Clause 19. The fluid dispenser system wherein the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

Clause 20. The fluid dispenser system wherein the medicant chamber segment, the nozzle and the container coupler segment are a monolithic unit.

Clause 21. The fluid dispenser system according to claim 1 wherein the nozzle and the container coupler segment are a monolithic unit, and is coupled to the medicant chamber segment.

Clause 22. The fluid dispenser system wherein the positioning arm is configured to depend from one of the medicant chamber segment, the nozzle or the container coupler segment, the positioning arm being selectively movable relative to the medicant chamber segment to deform the medicant chamber segment to pressurize the internal volume to facilitate distribution of the medicants through the nozzle passage and out the nozzle port.

Clause 23. A fluid dispenser system, which comprises:
a nozzle assembly configured for coupling to a container configured to hold medicants for treatment of one or more conditions of an eye of a subject, the nozzle assembly including:
a container coupler segment configured to engage with the container; a medicant chamber segment defining an internal volume configured to receive medicants from the container;
a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment;
a positioning arm including an engagement segment displaced from the medicant chamber segment, the engagement segment configured to engage at least one of an upper or lower eye lid surrounding the eye to stabilize the one eye lid to facilitate distribution of the medicants into the eye;
a medicant seal adjacent the medicant chamber segment, the medicant seal permitting passage of the medicants from the container into the internal volume of the medicant chamber segment; and
an air inlet port configured to enable entry of air into the medicant chamber segment.

Clause 24. The fluid dispenser system including a filter in communication with the air inlet port for filtering air entering into the container.

Clause 25. The fluid dispenser system wherein the positioning arm is configured to depend from one of the medicant chamber segment, the nozzle or the container coupler segment, the positioning arm being selectively movable relative to the medicant chamber segment to deform the medicant chamber segment to impart pressure into the internal volume to facilitate distribution of the medicants through the nozzle passage and out the nozzle port.

Clause 26. The fluid dispenser system wherein the medicant seal includes one or more seal passages to permit passage of fluids therethrough.

Clause 27. The fluid dispenser system wherein a first seal passage of the medicant seal is configured to permit passage of the medicants into the internal volume of the medicant chamber segment and a second seal passage of the medicant seal is configured to permit passage of air into the container to facilitate equalization of pressure inside the container.

Clause 28. The fluid dispenser system wherein the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

Clause 29. The fluid dispenser system including a reciprocating nozzle valve at least partially disposed in the nozzle passage of the nozzle, the reciprocating nozzle valve configured to selectively move in the nozzle port between an open condition to permit passage of the medicants out the nozzle port and a closed condition to selectively permit flow of medicants through the nozzle port.

### II. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’or ‘steps’of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise.

Headings of sections provided in this patent application and the title of this patent application are for convenience only, and are not to be taken as limiting the disclosure in any way.

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximating unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

It is within the scope of this disclosure that one or more of the various features described herein can be used with one or more of the various embodiments described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A fluid dispenser system, which comprises:
a nozzle assembly configured for coupling to a container configured to hold medicants for treatment of one or more conditions of an eye of a subject, the nozzle assembly including:
a container coupler segment configured to engage with the container;
a medicant chamber segment defining an internal volume configured to receive medicants from the container;
a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment;
a positioning arm including an engagement segment displaced from the medicant chamber segment, the engagement segment configured to engage at least one of an upper or lower eye lid surrounding the eye to stabilize the one eye lid to facilitate distribution of the medicants into the eye; and
a reciprocating nozzle valve at least partially disposed in the nozzle passage of the nozzle, the reciprocating nozzle valve configured to selectively move in the nozzle port to selectively permit and prevent flow of medicants through the nozzle passage and out the nozzle port.

2. The fluid dispenser system according to claim 1 wherein the reciprocating nozzle valve is configured to move in a first direction toward the nozzle port in response to pressurization of the internal volume of the medicant chamber segment to control flow of the medicants through the nozzle port, and in a second direction away from the nozzle port to close the nozzle passage in response to release of pressurization of the internal volume of the medicant chamber segment.

3. The fluid dispenser system according to claim 2 including a cannulated nozzle component disposed in the nozzle passage of the nozzle for receiving the reciprocating nozzle valve, and permitting movement of the reciprocating nozzle valve.

4. The fluid dispenser system according to claim 3 wherein the reciprocating nozzle valve and the cannulated nozzle component define a passageway therebetween during movement of the reciprocating nozzle valve in the first direction to permit passage of the medicants out the nozzle port.

5. The fluid dispenser system according to claim 4 wherein the reciprocating nozzle valve and the cannulated nozzle component include cooperating structure to close the passageway during movement of the reciprocating nozzle valve in the second direction.

6. The fluid dispenser system according to claim 5 wherein the cooperating structure include respective tapered portions of the reciprocating nozzle valve and the cannulated nozzle component.

7. The fluid dispenser system according to claim 2 including a spring disposed in the nozzle passage for biasing the reciprocating nozzle valve in the second direction to close the nozzle passage.

8. The fluid dispenser system according to claim 2 wherein the reciprocating nozzle valve includes outer grooves, the outer grooves permitting passage of the medicants through the nozzle port when the reciprocating nozzle valve is moved in the first direction.

9. The fluid dispenser system according to claim 1 an air inlet port in fluid communication with the medicant chamber segment to enable ingress of air into the medicant chamber segment.

10. The fluid dispenser system according to claim 9 including a filter in communication with the air inlet port for filtering air.

11. The fluid dispenser system according to claim 9 including a reciprocating pressurization valve at least partially disposed in an air inlet passage of the air inlet port, the reciprocating pressurization valve configured to selectively move in the air inlet port to selectively permit and prevent flow of air into the medicant chamber segment and relative to the container.

12. The fluid dispenser system according to claim 11 including a cannulated pressurization component disposed in the air inlet port for receiving the reciprocating pressurization valve, and permitting movement of the reciprocating pressurization valve.

13. The fluid dispenser system according to claim 12 wherein the reciprocating pressurization valve and the cannulated pressurization component define a passageway therebetween during movement of the reciprocating pressurization valve in a first direction to permit entry and passage of air though the air inlet port.

14. The fluid dispenser system according to claim 13 wherein the reciprocating pressurization valve and the cannulated pressurization component include cooperating structure to close the passageway during movement of the reciprocating pressurization valve in a second direction.

15. The fluid dispenser system according to claim 14 wherein the cooperating structure include respective tapered portions of the reciprocating pressurization valve and the cannulated pressurization component.

16. The fluid dispenser system according to claim 1 including a medicant seal disposed between the internal volume of the medicant chamber segment and the container.

17. The fluid dispenser system according to claim 16 wherein the medicant seal includes one or more seal passages to permit passage of fluids therethrough.

18. The fluid dispenser system according to claim 17 wherein a first seal passage of the medicant seal is configured to permit passage of the medicants into the internal volume of the medicant chamber segment and a second seal passage of the medicant seal is configured to permit passage of air into the container to facilitate equalization of pressure inside the container.

19. The fluid dispenser system according to claim 17 wherein the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

20. The fluid dispenser system according to claim 1 wherein the medicant chamber segment, the nozzle and the container coupler segment are a monolithic unit.

21. The fluid dispenser system according to claim 1 wherein the nozzle and the container coupler segment are a monolithic unit, and is coupled to the medicant chamber segment.

22. The fluid dispenser system according to claim 1 wherein the positioning arm is configured to depend from one of the medicant chamber segment, the nozzle or the container coupler segment, the positioning arm being selectively movable relative to the medicant chamber segment to deform the medicant chamber segment to pressurize the internal volume to facilitate distribution of the medicants through the nozzle passage and out the nozzle port.

23. A fluid dispenser system, which comprises:
a nozzle assembly configured for coupling to a container configured to hold medicants for treatment of one or more conditions of an eye of a subject, the nozzle assembly including:
a container coupler segment configured to engage with the container;
a medicant chamber segment defining an internal volume configured to receive medicants from the container;
a nozzle depending from the medicant chamber segment, the nozzle defining a nozzle passage in fluid communication with the medicant chamber segment and having a nozzle port for distribution of the medicants from the nozzle passage in response to pressurization of the internal volume of the medicant chamber segment;
a positioning arm including an engagement segment displaced from the medicant chamber segment, the engagement segment configured to engage at least one of an upper or lower eye lid surrounding the eye to stabilize the one eye lid to facilitate distribution of the medicants into the eye;
a medicant seal adjacent the medicant chamber segment, the medicant seal permitting passage of the medicants from the container into the internal volume of the medicant chamber segment; and
an air inlet port configured to enable entry of air into the medicant chamber segment.

24. The fluid dispenser system according to claim 23 including a filter in communication with the air inlet port for filtering air entering into the container.

25. The fluid dispenser system according to claim 23 wherein the positioning arm is configured to depend from one of the medicant chamber segment, the nozzle or the container coupler segment, the positioning arm being selectively movable relative to the medicant chamber segment to deform the medicant chamber segment to impart pressure into the internal volume to facilitate distribution of the medicants through the nozzle passage and out the nozzle port.

26. The fluid dispenser system according to claim 23 wherein the medicant seal includes one or more seal passages to permit passage of fluids therethrough.

27. The fluid dispenser system according to claim 26 wherein a first seal passage of the medicant seal is configured to permit passage of the medicants into the internal volume of the medicant chamber segment and a second seal passage of the medicant seal is configured to permit passage of air into the container to facilitate equalization of pressure inside the container.

28. The fluid dispenser system according to claim 27 wherein the medicant seal includes a first valve segment and a second valve segment arranged relative to the first valve segment, the first valve segment defining a first valve opening to permit passage of medicants from the container, and the second valve segment defining a second valve flap configured to be received in the first valve opening of the first valve segment, the second valve flap configured to at least partially close the first valve opening in response to pressurization of the internal volume of the medicant chamber segment.

29. The fluid dispenser system according to claim 23 including a reciprocating nozzle valve at least partially disposed in the nozzle passage of the nozzle, the reciprocating nozzle valve configured to selectively move in the nozzle port between an open condition to permit passage of the medicants out the nozzle port and a closed condition to selectively permit flow of medicants through the nozzle port.
